(19) United States
(12) Patent Application Publication
(10) Pub. No.: US 2026/0041093 A1
(43) Pub. Date: Feb. 12, 2026

(54) Ionic Derivatives Of Aromatic Carboxylic Acid For Use As Plant Stimulants, Method For Stimulating Plants And Use Of These Derivatives For Manufacturing Compositions For Stimulating Plants

(71) Applicants: FUNDACJA UNIWERSYTETU IM. ADAMA MICKIEWICZA W POZNANIU, Poznan (PL); INNOSIL SPOLKA Z OGRANICZONA ODPOWIEDZIALNOSCIA, Poznan (PL)

(72) Inventors: Marcin SMIGLAK, Poznan (PL); Henryk POSPIESZNY, Poznan (PL); Rafal KUKAWKA, Krzyszkowo (PL); Maciej SPYCHALSKI, Poznan (PL)

(73) Assignees: FUNDACJA UNIWERSYTETU IM. ADAMA MICKIEWICZA W POZNANIU, Poznan (PL); INNOSIL SPOLKA Z OGRANICZONA ODPOWIEDZIALNOSCIA, Poznan (PL)

(21) Appl. No.: 19/154,672

(22) PCT Filed: Dec. 29, 2023
(86) PCT No.: PCT/PL2023/050110

§ 371 Date: Aug. 7, 2025

(30) Foreign Application Priority Data
Dec. 30, 2022 (PL) 443354
Apr. 5, 2023 (PL) 444328

(51) Int. Cl. A01N 43/82; A01N 33/12; A01N 37/10; A01P 21/00; C07C 65/10; C07C 211/63; C07D 285/14

(52) U.S. Cl. CPC A01N 43/82; A01N 33/12; A01N 37/10; A01P 21/00; C07C 65/10; C07C 211/63; C07D 285/14

(57) ABSTRACT

The invention relates to aromatic carboxylic acid ionic derivatives, wherein anion is represented by a general formula (I) or (II), and cation is selected from cation M+ and a cation of general formula (III), wherein the formulae (I), (II) and (III) are defined in the description, for use as a plant stimulant in plant growth regulation and/or in regulation of plant metabolic processes, and also—in case of ionic derivatives, wherein the anion is defined by the general formula (II)—for preventing the effects of biotic stress caused by the activity of viruses, bacteria and/or fungi. The invention also relates to a plant stimulation method and use of aromatic carboxylic acid ionic derivatives as active compounds for the production of compositions for stimulating plants by regulating their growth and/or metabolic processes, and in case of derivatives, wherein the anion is defined by the general formula (II)—also for preventing the effects of biotic stress caused by the activity of viruses, bacteria and/or fungi.

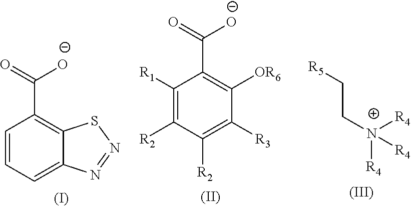

### FIELD OF THE INVENTION

The invention concerns ionic derivatives of carboxylic acid for use as plant stimulants in plant growth regulation and/or in regulation of plant metabolic processes as well as for preventing the effects of a biotic stress caused by the activity of viruses, bacteria and/or fungi. The invention also relates to a plant stimulation method by regulating their growth and/or their metabolic processes and/or by preventing the effects of a biotic stress.

### STATE OF THE ART

The plant resistance to environmental factors, determined by their defense mechanisms supporting plant survival under stress conditions, may be constitutive or inducible. In the first case, the defense mechanisms act throughout the entire plant life. The inducible resistance is related to the effects of stress factors, known as stressors. Environmental stress factors may be biotic or abiotic. The abiotic stressors include temperature (high and low, including frost), excessive or insufficient light radiation, drought, lack of oxygen, mechanical factors (wind, snow cover, ice cover) and chemical compounds (salinity, toxins, microplastics, flotation tailing and mineral deficiency). Biotic stressors include microorganisms (fungi and bacteria), viruses, plants (allelopathy, parasitism and competition) and animals (biting, parasitism and trampling). The search for solutions that would ensure the best possible conditions for plant growth and development, for example by protecting plants from various biotic and abiotic stressors, leading to increased yields, is continuously underway. Higher yields and improved quality of crops are strived for by farmers by providing plants with optimal growth conditions through all known agrotechnical means (such as ploughing, crop rotation, fertilization, irrigation and protection against diseases, pests and weeds).

Literature reports two main types of plant defense: induced and constitutive. In case of induced resistance, interaction with a pathogen leads to activation of relevant signaling pathways—hormones—that either induce or extinguish the production of genes responsible for a response to a given type of stress. For example, an increase in salicylic acid (SA) production has been shown to be induced by pathogenic infection or administration of certain substances, e.g. benzothiadiazole derivatives. An increase in SA results in the production of pathogenesis-related (PR) genes, which ultimately increase plant resistance to plant pathogens. This is a phenomenon of induced plant resistance, the mechanism of which is different from that involved when using stimulants.

Substances with stimulating effects are used in plant cultivation to improve growth and development processes. It is believed that the effect of stimulants on plants is due to their effect on metabolism rather than their direct involvement in the regulation of vital processes. In case of protecting a plant from the effects of abiotic stress, administration of a stimulant gives a signal to initiate the production of hormones responsible, e.g. for root development (to prevent the effects of drought), or for acceleration of chlorophyll production (resulting in accumulating more green mass in the plant). In case of preventing the effects of biotic stress, after the attack of a pathogen or administration of a substance (growth stimulant), plant mechanisms produce a number of substances such as polyphenols, anthocyanins, terpenoids, which interfere with the metabolism of pathogens once they enter the plant, delay or completely prevent pathogen reproduction or development, or repel pathogens, preventing the effects of biotic stress. For example, administration of stimulants increases the content of polyphenols and anthocyanins in a plant (while not resulting in an increase of salicylic acid production in the plant), making it very difficult for the pathogen to develop in the plant. Use of stimulants in plant cultivation can increase crop yields, often also improving crop quality. Stimulants can also improve life processes in plants without changing their natural behavior. The effects induced by the use of growth stimulants include: an increase in root mass, an increase in fresh and dry mass of the aboveground parts, an increase in the number of shoots, an increase in the plant height parameter, plant diameter, yield, the content of health-promoting substances in the green parts, roots and fruit, better use/absorption of active (mineral) substances from soil, better use/uptake of a fertilizer, an increase in chlorophyll content, higher photosynthesis levels, plant growth regulation, as well as prevention of the effects of abiotic stress caused by e.g. drought, herbicides and low temperature.

Plant stimulants can stimulate the synthesis of natural hormones and sometimes increase their activity, improve the uptake of minerals from soil or regulate root growth. In addition, they can increase resistance to adverse conditions, including abiotic stresses and the activity of pathogenic viruses, bacteria and fungi (biotic stress).

A number of solutions aimed at stimulation of plants in different aspects of their growth, have been proposed.

US 2005/0050587 A1 discloses use of 3-chlorosalicylic acid to induce production of PR-1 proteins to increase plant resistance to TMV virus and to act as a plant resistance inducer. However, this solution does not address the stimulation of a plant's metabolic processes to prevent the effects of stress, but indirectly limits the action of the stress factor. Moreover, the solution concerns only the neutral molecule of 3-chlorosalicylic acid, not its ionic derivatives.

U.S. Pat. No. 4,931,581 discloses a method and composition for artificially inducing in plants defensive mechanisms against diseases (plant immunity induction) and means and substances for completing this method.

DE 102008006622 A1 discloses use of benzothiadiazoles for protecting plants against the effects of cold, whereby the solution described concerns only the neutral (non-ionic) substances.

U.S. Pat. No. 8,754,011 B2 discloses methods and compositions for improving plant health by using a known herbicide, 3,6-dichloro-2-methoxybenzoic acid (dicamba) and its derivatives for a defense against biotic and abiotic stress, in particular the stress caused by exposure to a herbicide.

U.S. Pat. No. 5,190,928 (1991), based on many examples, describes the activity of substances—benzothiadiazole derivatives in plant-pathogen models as substances immunizing plants against plant diseases caused by microorganisms. The application is carried out by different methods, e.g. by administration through leaves, through soil and by seed treatment. Plant-pathogen models presented as examples, include Colletotrichum lagenarum on Cucimis sativus L. (cucumber), Pyricularia oryzae on rice, Pseudomonas lachrymans on Cucimis sativus L. (cucumber), Xanthomonas oryzae on rice, Xanthomonas vesicatoria on pepper, Phytophthora infectants on tomato, Plasmopara viticola on grape, Pseudomonas tomato on tomato, Phytophthora parasitica var. nicotiniae on tobacco, Peronospora tabacina on tobacco, Cercospora nicotianae on tobacco, Pseudomonas tabaci on tobacco, Erysiphe graminis on wheat.

B. Guichard et al. Pest Manag Sci 2022; 78: 4913-4928 describe a synthesis, phloem mobility and induced resistance of plants against synthetic conjugates of amino acids of salicylic acid or glucose, stimulating plant defensive reactions against pathogens, intermediated by salicylic acid. This publication confirms the activity of salicylic acid and its (neutral) derivatives as plant resistance inducers.

L. Klepper et al. Pest. Biochem. Physiol. 1991, 39, 43-48 show that halogen derivatives and other highly active salicylic acid derivatives can act as herbicide synergists when used in combination with a photosynthesis-inhibiting herbicide or can be sufficiently active and stable within the plant leaf tissue to act as herbicides when used alone. This publication focuses on the herbicidal (plant-damaging) effects of salicylic acid and its (neutral) derivatives.

U.S. Pat. Nos. 5,190,928 and 5,523,311 describe compositions containing benzo[1,2,3]thiadiazole derivatives to be used for the protection (immunization process) of plants against attack by phytopathogenic microorganisms or viruses.

PL232617B1, PL231217B1 and PL230659B1 disclose use of anionic derivatives of 7-carboxybenzo[1,2,3]thiadiazole acid (BTH), showing biological activities including antibacterial, antifungal, insecticidal, repellent and plant growth regulation. The group of counterions used includes, among others, choline. Plant growth regulators can be understood as substances of diverse chemical structure, which in plant organisms initiate or modify the course of life processes, including plant development. However, these patents refer to use of such compounds in plant protection by inducing systemic plant resistance (SAR). The examples of implementation disclosed in these documents merely show the steps of synthesis of a target compound in form of a 7-carboxybenzo[1,2,3]thiadiazole acid derivative and studies related to induction of plant immunity, antibacterial properties and physical properties.

On the other hand, U.S. Pat. No. 6,770,593B1 and US20040035162A1 describe compositions of fertilizing and antifungal activity, their syntheses and methods of plant fertilization and fungi control using these substances. The disclosed fertilizing compositions contain at least one phosphonate and at least one thiosulphate, as well as a synergistically acting salicylic acid, its homolog (benzoic acid), derivative (salicylamide or esters) or salt (potassium or sodium salicylate). These compositions are useful as fertilizers, especially for foliar use. They increase plant growth to a greater degree than each individual component applied alone, stimulate plant growth and/or influence crop yield, for example by reducing tuber blight. Furthermore, in a preferred variant, the composition comprises a plant growth regulator, preferably chlormequat.

There is an ongoing need for compounds that could stimulate plant growth under standard growing conditions as well as under abiotic and biotic stress conditions. In particular, there is a need for compounds suitable for use as plant stimulants in regulating plant growth and/or in regulating plant metabolic processes and/or preventing the effects of biotic stress.

### Summary of the Gist of the Invention

The subject of the invention in the first aspect is an aromatic carboxylic acid ionic derivative, wherein anion is defined by a general formula (I) or (II),

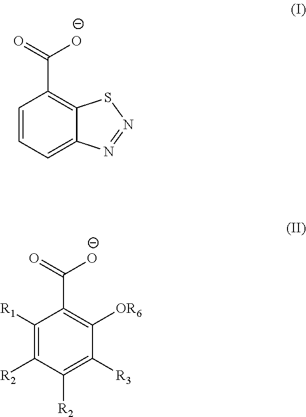

while cation is either a cation M+ or a cation of a general formula (III)

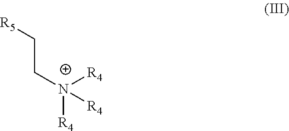

where
R₁ denotes H, F, Br or I,
R₂ and R₃, independently, denote H, F, Cl, Br or I,
R₄ denotes methyl, ethyl, propyl, butyl, decyl or dodecyl,
R₅ denotes hydroxyl, methoxyl, ethoxyl, H, F, Cl, Br, I;
R₆ denotes H, methyl, ethyl or acetyl,
M⁺ denotes lithium, sodium, potassium, calcium or magnesium cation, whereby in case of a divalent cation there are two acid residues per such cation;
   for use as a plant stimulant for plant growth regulation and/or regulation of plant metabolic processes.

Preferably, M⁺ denotes sodium or potassium cation, R₁ denotes H, R₂ and R₃, independently, denote H or Cl, R₄ denotes methyl, R₅ denotes hydroxyl, while R₆ denotes H. It is particularly preferred, when the above-defined aromatic carboxylic acid ionic derivative is selected from choline 3-chlorosalicylate, choline 5-chlorosalicylate, choline 3,5-dichlorosalicylate and choline benzo[1,2,3]thiadiazole-7-carboxylate.

Preferably, the plant stimulant is selected from: a stimulant of root mass growth, a stimulant of growth of fresh and/or dry mass of aboveground part, a stimulant of increase in shoot number, a stimulant of increase in plant height and/or diameter, a stimulant of increase in yield, a stimulant of increase in the content of health-promoting substances in the green parts, roots and fruit, a stimulant of increase in the chlorophyll content, a stimulant of increase in photosynthesis level, a stimulant of increase in the uptake efficiency of active substances (minerals) from soil, a stimulant of increase in fertilizer uptake efficiency.

The subject of invention in the second aspect is an aromatic carboxylic acid ionic derivative, wherein anion is defined by the general formula (II), while cation is selected from a cation M+ and a cation of the general formula (III), whereby the general formula (II), cation M⁺ and the general formula (III) are defined above for use as a plant stimulant for preventing the effects of biotic stress effected by activity of viruses, bacteria or/and fungi.

Also in this aspect of invention, M⁺ preferably denotes a cation of sodium or potassium, R₁ denotes H, R₂ and R₃, independently, denote H or Cl, R₄ denotes methyl, R₅ denotes hydroxyl, and R₆ denotes H. Particularly preferred aromatic carboxylic acid ionic derivative defined above with respect to the second aspect of the invention is selected from choline 3-chlorosalicylate, choline 5-chlorosalicylate and choline 3,5-dichlorosalicylate.

The subject of invention in the third aspect is a plant stimulation method involving exposing said plant to a composition containing at least one active ingredient, wherein the aromatic carboxylic acid ionic derivative defined above when presenting the first aspect of invention, is used as the active ingredient.

Preferably, choline 3-chlorosalicylate, choline 5-chlorosalicylate and choline 3,5-dichlorosalicylate or choline benzo[1,2,3]thiadiazole-7-carboxylate is used as the active ingredient of the composition.

Preferably, the composition containing the active ingredient is applied to the plant roots and/or leaves and/or seeds, in particular once every 5 to 21 days.

Preferably, the composition containing the active ingredient is applied to plants subjected to abiotic stress factors, including drought, too high or too low temperatures or use of herbicides.

Preferably, the composition in form of a solution, preferably an aqueous solution, containing the active ingredient is applied to the plants by spraying or watering.

The subject of the invention in the fourth aspect is use of at least one aromatic carboxylic acid ionic derivative defined above when presenting the first aspect of invention as at least one active ingredient for production of a composition for plant stimulation by regulation of their height and/or metabolic processes.

Preferably, the aromatic carboxylic acid ionic derivative is selected from choline 3-chlorosalicylate, choline 5-chlorosalicylate, choline 3,5-dichlorosalicylate and choline benzo[1,2,3]thiadiazolo-7-carboxylane. In this use it is particularly preferred that the aromatic carboxylic acid ionic derivative is the sole active ingredient in the composition for plant stimulation. In selected embodiments of this aspect of the invention it is possible to use in the composition other active ingredients such as, for example, fungicides, herbicides or other stimulants.

The subject of the invention in the fifth aspect is use of at least one aromatic carboxylic acid ionic derivative defined above with respect to the second aspect of the invention as at least one active ingredient for production of a composition for plant stimulation by preventing the effects of biotic stress caused by the activity of viruses, bacteria and/or fungi.

Preferably, the aromatic carboxylic acid ionic derivative is selected from choline 3-chlorosalicylate, choline 5-chlorosalicylate and choline 3,5-dichlorosalicylate. In this use it is particularly preferred that the aromatic carboxylic acid ionic derivative is the sole active ingredient in the composition for plant stimulation. In selected embodiments of this aspect of the invention it is possible to use in the composition other active ingredients such as, for example, fungicides, herbicides or other stimulants.

The effects of plant stimulation by contact with an aromatic carboxylic acid ionic derivative defined above with respect to the first aspect of the invention, include: increased root mass, increased fresh and dry mass of the above-ground parts, increased number of shoots (e.g. in the case of apple trees), increased plant height or diameter, increased yield, increased content of health-promoting substances in the green parts, roots and fruit (e.g. lycopene and sugar content which is expressed by Brix index, in tomatoes, protein and gluten in wheat, oil components in rapeseed); improved use/uptake of active substances (minerals) from soil; improved fertilizer uptake; increased chlorophyll content; increased photosynthesis (lettuce); prevention and/or alleviation of abiotic stress caused by drought (lettuce), herbicides and low temperature; plant growth regulation.

### BRIEF DESCRIPTION OF FIGURES

The invention in exemplary embodiments is illustrated in the drawings, wherein:

FIG. 1 shows a photo of tomato plants of Olga cultivar treated three times with a solution of choline benzo[1,2,3[thiadiazole-7-carboxylate at a concentration of 25 mg/L (left side) and control plants treated with distilled water (right side).

FIG. 2 shows a photo of broccoli plants treated once with a 5 mg/L choline benzo[1,2,3[thiadiazole-7-carboxylate solution (right side of photo) and control plants treated with distilled water (left side).

FIG. 3 shows a photo of a Star of Bethlehem plants treated three times with a 50 mg/L choline 3-chlorosalicylate solution (two plants on the left) and control plants treated with distilled water (two plants on the right).

FIG. 4 shows a photo of tulips: left side—untreated control (UTC), the middle—the plants treated with choline 3-chlorosalicylate, right side—plants treated with choline benzo[1,2,3]thiadiazole-7-carboxylate.

FIG. 5 shows a photo of a tobacco plant (Nicotiana tabacum) cv. Xanthi watered twice with a solution of the choline 3-chlorosalicylate at a concentration of 80 mg/L (right side) and control plants treated with distilled water (left side).

### DETAILED DESCRIPTION OF THE INVENTION

The invention in the first aspect concerns ionic derivatives of aromatic carboxylic acids, in particular 7-carboxybenzo[1,2,3]thiadiazole acid and salicylic acid, in form of salt with an inorganic cation such as a metal cation of groups I and II of the periodic table (Na⁺, K⁺, Mg²⁺, Ca²⁺) or in form of salt with an organic tetraalkylammonium cation, in particular a choline cation (trimethyl-ethyleneammonium cation) for use as plant stimulants in plant growth regulation and/or in regulation of plant metabolic processes.

The second aspect of the invention concerns ionic derivatives of aromatic carboxylic acids, in particular salicylic acid, in form of salt with an inorganic cation such as a metal cation of groups I and II of the periodic table (Na⁺, K⁺, Mg²⁺, Ca²⁺) or in form of salt with an organic tetraalkylammonium cation, in particular a choline cation (trimethyl-ethyleneammonium cation) for use in preventing the effects of biotic stress caused by activity of viruses, bacteria and/or fungi.

The third aspect of the invention concerns a plant stimulation method involving exposing said plant to a composition containing at least one active ingredient, whereby the aromatic carboxylic acid ionic derivative defined above with respect to the first aspect of the invention, is used as the active ingredient. The fourth aspect of the invention concerns use of at least one ionic derivative of an aromatic carboxylic acid, as defined above with respect to the first aspect of the invention, as at least one active ingredient for production of a composition for plant stimulation by regulation of their height and/or metabolic processes. The fifth aspect of the invention concerns use of at least one ionic derivative of an aromatic carboxylic acid, as defined above with respect to the second aspect of the invention, as at least one active ingredient for production of a composition for plant stimulation by preventing the effects of biotic stress caused by the activity of viruses, bacteria and/or fungi.

According to the present invention, a composition for stimulating plants by regulating their growth and/or their metabolic processes (according to the first aspect) or for preventing the effects of biotic stress (according to the second aspect) may contain an ionic derivative of an aromatic carboxylic acid as defined above in an agriculturally effective amount, in particular in a concentration of 0.001 to 1000 mg/l, for example 0.04 to 400 mg/i.

According to the present invention, the compositions may be in any suitable form for application to a plant or to soil in the immediate vicinity of a plant. In some aspects of the invention, the compositions may be in form of an aqueous solution, a solution in an organic solvent such as alcohol, a mixture containing inorganic and organic solvents such as a mixture of water and alcohol, or in form of an emulsion. If the compositions contain a mixture of inorganic and organic solvents, the solvents may be in a ratio of between 1:1000 and 1000:1. If the composition contains a mixture of water and alcohol, water may be present in an amount of between 0.01% and 100% by volume of the mixture. On the other hand, when the composition is in form of an emulsion, then the active compound=ionic derivative of the aromatic carboxylic acid defined above may be encapsulated or be in form of a suspension in that emulsion.

According to the presented invention, the compositions may additionally comprise an adjuvant. The adjuvant may be present in an amount of 10% by volume or less of the total volume of the composition. In some aspects of the invention, the compositions may further comprise a fungicide, an antiviral agent or an antimicrobial agent, or may comprise a composition of an organic or mineral fertilizer or other plant stimulant.

According to the presented invention, the compositions comprising ionic derivatives of aromatic carboxylic acid, as defined above with respect to the first aspect of the invention, are used as plant growth stimulants, for example to regulate plant growth, to regulate metabolic processes of a plant, to regulate physiological processes of a plant or to prevent the effects of abiotic stress in a plant, with the achievement of at least one of the following effects: an increase in root mass or in fresh and dry mass of the aboveground parts or in number of shoots, height or diameter of a plant, in yield or in content of health-promoting substances in green parts, roots and fruit, in chlorophyll content or a higher level of photosynthesis or a more efficient use/uptake of active (mineral) substances from soil or a more efficient fertilizer uptake.

The presented invention also discloses methods for using the compositions. The methods may include exposing plant seeds, root or leaves to the disclosed compounds or compositions. In some aspects, the compositions may be administered to the roots by soil spraying, mechanical introduction, mixing with fertilizer, soil improvement or the like. The compositions may be administered periodically. In some aspects, the compositions may be administered to a plant from 1 to 5 times. In some aspects, the compositions may be administered once per week.

Depending on the intended mode of administration, the compositions described in the presented invention may be in form of a solid, semi-solid, liquid, solution, suspension, emulsion, gel, oil dispersion, capsule (such as an active ingredient encapsulated in a microcapsule) or the like. The compositions may comprise, as mentioned above, an agriculturally effective amount of the active ingredient, that is the ionic derivative of aromatic carboxylic acid, as defined above, in combination with an agriculturally acceptable carrier, and may further comprise other carriers, adjuvants, diluents, thickeners, buffers, preservatives, surfactants, and the like. In some aspects certain concentrates of the compositions for diluting, which in addition to water contain compositions of moisturizing agent, binder, dispersing agent or emulsifier.

Agriculturally acceptable carrier can include an organic or inorganic carrier. Exemplary carriers include, but not exclusively, water, organic solvents, inorganic solvents, petroleum fractions or hydrocarbons such as mineral oils, aromatic solvents, paraffin oils, vegetable oils such as soy oil, rapeseed oil, olive oil, castor oil, sunflower oil, coconut oil, corn oil, cotton seed oil, linseed oil, palm oil, arachid oil, safflower oil, sesame oil, tung oil, esters of above vegetable oils, esters of mono-, di- or trihydric alcohols or other lower polyhydric alcohols (i.e. containing 4-6 hydroxy groups), such as 2-ethylhexyl stearate, n-butyl oleate, isopropyl myristate, propylene glycol dioleate, dioctyl succinate, dibutyl adipate, dioctyl phthalate, mono-, di- and polycarboxylic acid esters, toluene, xylene, naphtha, vegetable oil, acetone, methyl ketone-ethyl, cyclohexanone, trichlorethylene, perchlorethylene, ethyl acetate, amyl acetate, butyl acetate, propylene glycol monomethyl ether and diethylene glycol monomethyl ether, methyl alcohol, ethyl alcohol, isopropyl alcohol, amyl alcohol, ethylene glycol, propylene glycol, glycerin, N-methyl-2-pyrrolidinone, N,N-dimethylalkylamides, dimethylsulfoxide, liquid fertilizers and their mixtures. Other exemplary carriers include silicas, silica gels, silicates, talc, kaolin, limestone, lime, chalk, pulement, loess, clay, dolomite, diatomaceous earth, calcium sulfate, magnesium sulfate, magnesium oxide, ground synthetic materials, pyrophyllite clay, attapulgite, diatomaceous earth, calcium carbonate, bentonite clay, Fuller's earth, cotton hulls, wheat flour, soy flour, pumice, wood flour, walnut shell flour, lignin, ammonium sulfate, ammonium phosphate, ammonium nitrate, ureas, grain meal, walnut shell meal tree bark, tree bark flour and nut shell flour, cellulose powders and mixtures thereof. The agriculturally acceptable carrier may be present in an amount of 99.9% by weight or less, 99% by weight or less, 98% by weight or less, 97% by weight or less, 95% by weight or less, 90% by weight or less, 85% by weight or less, 80% by weight or less, 75% by weight or less, 70% by weight or less, 65% by weight or less, 60% by weight or less, 55% by weight or less, 50% by weight or less, 45% by weight or less or 40% by weight or less based on the total weight of the composition.

Examples of agriculturally acceptable adjuvants include, but are not limited to, antifreezes, antifoams, compatibilizing agents, masking agents, neutralizing agents and buffers, corrosion inhibitors, dyes, fragrances, penetration agents, wetting agents, spreading agents, dispersants, thickening agents, freezing point depressants, antimicrobial agents, vegetable oil, protective agents, adhesive agents, surfactants, protective colloids, emulsifiers, tackifiers and mixtures thereof. The agriculturally acceptable adjuvant may be present in an amount of 15% by volume or less, 10% by volume or less, or 5% by volume or less, based on the total volume of the composition.

According to the present invention, the compositions may contain from 0.001 to 99% by weight of the active compound, i.e. an ionic derivative of an aromatic carboxylic acid as defined above, together with carriers and/or adjuvants. In some embodiments, the compositions may be in the form of a solution having an active compound concentration of 0.001 mg/L or greater. For example, the compositions may contain from 0.001 mg/l to 900 mg/l, from 0.01 mg/l to 800 mg/l, from 0.01 mg/l to 700 mg/l, from 0.01 mg/l to 500 mg/l, from 0.01 mg/l to 300 mg/l, from 0.01 mg/l to 100 mg/l, from 0.1 mg/l to 500 mg/l, from 0.1 mg/1 to 300 mg/l, from 0.1 mg/l to 200 mg/l or from 0.1 mg/l to 100 mg/l of the active compound. In some embodiments, the compositions may be in the form of a solution having an active compound concentration of 900 mg/L or less.

As used herein, the term “plant” includes whole plants and parts thereof, including, but not limited to, shoot organs/vegetative structures (e.g., leaves, stems and tubers), roots, flowers, and floral organs/structures (e.g., bracts, sepals, petals, stamens, pistils, anthers and ovules), seeds (including embryo, endosperm and seed coat) and fruits (mature ovary), plant tissue (e.g. vascular tissue, ground tissue, etc.) and cells (e.g. cells protective cells, ova and the like) and their offspring. The classes of plants to which the solutions of the present invention may be applied include classes of higher and lower plants, including angiosperms (monocotyledonous and dicotyledonous plants), gymnosperms, ferns, horsetails, psilophytes, lycophytes, bryophytes and multicellular algae. For example, plants to which the present invention may be used include any vascular plant, for example monocotyledonous or dicotyledonous plant or gymnosperm, including, but not limited to, alfalfa, apple, radish, banana, barley, rapeseed, castor bean, chrysanthemum, clover, cocoa, coffee, cotton, cottonseed, corn, blueberry, cranberry, cucumber, dendrobium, yam, eucalyptus, fescue, flax, gladiolus, lilac, linseed, millet, cucumber melon, mustard, oats, oil palm, rapeseed, papaya, peanuts, pineapple, ornamental plants, beans, potato, rapeseed, rice, rye, ryegrass, safflower, sesame, sorghum, soybean, sugar beet, sugar cane, sunflower, strawberry, tobacco, tomato, turf grass, wheat and vegetables, such as lettuce, celery, broccoli, cauliflower, cucurbits, onions (including garlic, shallots, leeks and chives); fruit and nut trees such as apple, pear, peach, orange, grapefruit, lemon, lime, almond, pecan, walnut (Juglans L.), hazel; vines such as grapes, kiwi, hops; fruit bushes and blackberries, e.g. raspberry, blackberry, gooseberry; forest trees such as ash, pine, fir, maple, oak, chestnut, common; with alfalfa, rapeseed, castor beans, corn, cotton, mud, flax, linseed, mustard, oil palm, rapeseed, peanuts, potatoes, rice, safflower, sesame, soybean, sugar beet, sunflower, tobacco, tomatoes and wheat. In some embodiments, plants to which the present invention may be used include any crop plant that is a fodder crop, an oilseed crop, a cereal crop, a fruit crop, a vegetable crop, a fiber crop, a spice crop, a nut crop, a peat crop, sugar cultivation, cultivation for beverages and forestry.

As used in this description, the term “plant stimulant” refers to a substance or micro-organism applied to plants in the conditions that enhance the plants nutritional efficiency, stress tolerance and/or yield quality characteristics, irrespective of their nutritional content. In particular, plant stimulants are used in plant cultivation to improve growth and development processes and to prevent the effects of stress caused by biotic agents such as viruses, bacteria and fungi. The effect of stimulants on plants is not related to their direct involvement in the regulation of vital processes, but to their influence on plant metabolism in a broad sense. They can stimulate the synthesis of natural hormones and sometimes increase their activity, improve the uptake of minerals from the soil, and regulate root growth. In addition, they can cause an increase in resistance to adverse conditions (biotic or abiotic). The use of stimulants in crop cultivation increases yields, often while also improving the crop quality. Stimulants can improve vital processes in plants without altering the natural plant behavior. The compounds described in this description are plant stimulants and can be used as plant growth regulators, regulators of plant metabolic processes, regulators of plant physiological processes, substances that prevent the effects of abiotic and biotic stresses in the plant. Compositions containing ionic derivatives of aromatic carboxylic acid as defined above, can be used as a plant stimulant for both healthy and unhealthy plants or plants growing in both healthy and unhealthy environments.

As mentioned above, U.S. Pat. Nos. 5,190,928 and 5,523,311 describe use of benzo[1,2,3]thiadiazole derivatives as plant immunizers against attack by phytopathogenic microorganisms or viruses. However, it has been unknown to use these compounds as plant stimulants, such as plant growth regulator, growth promoter causing an increase of root weight, increase of fresh and dry mass of the aboveground parts, increase of number of shoots, increase of height parameter, plant diameter, increase of yield, increase of content of health-promoting substances in green parts, roots and fruit, improved use/uptake of active (mineral) substances from soil, improved use/uptake of fertilizer, increased chlorophyll content, higher photosynthesis, prevention of the effects of abiotic stress caused by drought, exposure to herbicides or low temperatures. In particular, the difference between an immunizing agent and a plant stimulant is clear to a person skilled in the field of the invention. For example, an immunizing agent is generally administered once to an organism (plant or animal) to provide resistance to a specific disease for the rest of the organism's life. In contrast, a plant stimulant is generally applied multiple times during the plant life, as it must continuously stimulate the plant in order for the plant to exhibit the desired function, for example every 5 to 14 days.

At the same time, according to common knowledge in the field of the invention, the modification of a neutral (non-ionic) molecule to a salt form leads to formation of a new chemical individual, with unknown and unpredictable physicochemical and biological properties. Derivatization of neutral molecules to a ionic form involves, among other things, an abrupt change in solubility of the chemical molecule in water and organic solvents, a change in melting point, decomposition point, electron conductivity in solutions, a change in pH of an aqueous solution, a change of ratio of dissolution in water to dissolution in n-octanol (logP). It is completely impossible to predict a derivatization that will ‘transfer’ biological properties from a neutral molecule to its ionic form. For this reason, the properties of ionic derivatives are not predictable and cannot be routinely deduced from information on the properties of neutral derivatives of the same compounds.

In addition, the ionic aromatic carboxylic acid derivatives defined above show previously unknown and unpredictable properties of stimulating plant growth and development. These properties allow to use these compounds as plant growth regulators, growth stimulants causing an increase of root mass, an increase of fresh and dry mass of the aboveground parts, an increase of number of shoots, an increase of height parameter, plant diameter, an increase of yield, an increase of content of health-promoting substances in green parts roots and fruit, an improved use/uptake of active substances (minerals) from soil, an improved fertilizer uptake, an increased chlorophyll content, a higher photosynthesis levels, a prevention of the effects of abiotic stress of drought, exposure to herbicides, or low temperatures.

In some aspects of the present invention, the compositions comprising ionic derivatives of aromatic carboxylic acid as defined above, can be used to stimulate and prevent the effects of biotic stress on seeds during germination, protect a plant from biotic stress induced by biotic stressors and abiotic stress induced by physical or chemical stressors of unanimated origin, such as the presence of harmful chemicals including salt, limited access to water, sunburn, wind, nutrient deficiency or improper cultivation practices such as over-watering or too deep planting.

As indicated above, one aspect of the present invention concerns methods of using the compositions containing ionic derivatives of aromatic carboxylic acid defined above, as plant stimulants. Such a method includes contacting the plant with an agriculturally effective amount of such a composition, wherein any part of the plant, including roots, flowers, leaves, stems and seeds, is contacted with the composition using any known technique. Exemplary techniques for contacting the compositions with plants include, but are not limited to, spraying, spraying, dusting, spreading, spraying, dripping, dipping, sprinkling, injecting, hydroponics, or direct water (in-water) application. The method of application may vary depending on the intended purpose. The compositions can be applied to plants in the field or in a greenhouse. In some aspects, the compositions may be applied to a part of the plant, such as tubers, prior to planting.

The composition may be contacted with any part of the plant, for example the root or leaves of the plant. In some embodiments, the composition may be contacted with the roots by soil spraying, mechanical application, mixing with fertilizer, soil amendment, premix, or the like.

The selected dosage level of a given composition will depend on various factors, including, for example, on the activity of the particular ionic aromatic carboxylic acid derivative defined above, the route of administration, the time of administration, the duration of treatment, other active substances and/or materials used in combination with the particular ionic aromatic carboxylic acid derivative used as defined above, the condition and general health of the treated plant and similar factors well known in the agriculture. However, the compositions described in the presented invention provide plant stimulation even at low doses. In some exemplary embodiments, the compositions can be applied at a dose ranging from 0.001 g active ingredient/ha to 1000 g active ingredient/ha. For example, the compositions can be applied at a rate from 0.02 g active substance/ha to 200 g active substance/ha.

According to the present invention, the compositions may be applied to plants in a periodic manner. In some aspects, the plant may be contacted with the composition two or more times. For example, the plant may be contacted with the composition 3, 4, 5, 6, 7, 8, 9 or 10 times. In some implementation examples, the plant may be contacted with the composition 2 to 5 times. In some implementation examples, the plant only needs to be contacted with the composition once. In some aspects, the plant may be contacted with the composition once every 5 to 21 days. For example, the plant can be contacted with the composition once every 5, 6, 7, 8, 9, 10, 11, 12, 13, 14, 15, 16, 17, 18, 19, 20 or 21 days. In some implementation examples, the plant may be contacted with the composition once per week. In some aspects, the plant may be contacted with the composition from 1 to 5 times over 5 to 21 days. For example, the plant may be contacted 1 to 5 times per week.

In some aspects, the compositions described herein may be used preventively, prior to the occurrence of the stress factor(s).

The compositions may be applied in combination with an additional plant protection agent. For example, the composition can be used with a fungicide, an antiviral agent or an antimicrobial agent. A composition comprising an ionic derivative of aromatic carboxylic acid, as defined above, and a fungicide, antiviral agent or antimicrobial agent may be applied to a plant simultaneously or sequentially. In some exemplary embodiments, the fungicide, antiviral agent or antimicrobial agent may be applied to a plant after the composition comprising the ionic aromatic carboxylic acid derivative as defined above.

### EXAMPLES

### Example 1: Effect of Selected Ionic Derivatives of Aromatic Carboxylic Acids on the Stimulation of Plant Height, Stem Diameter, Growth of Fresh and Dry Weight of the Aboveground Part, Growth of Fresh and Dry Weight of Roots, Root Length, Flowering Length and Vigor of Tulips

Tulip bulbs planted in clean peat medium were watered with 10 to 50 mL of a 30 mg/L working solution containing 1. choline benzo[1,2,3]thiadiazole-7-carboxylate; 2. sodium benzo[1,2,3]thiadiazole-7-carboxylate; 3. potassium benzo[1,2,3]thiadiazole-7-carboxylate; 4. magnesium benzo[1,2,3]thiadiazole-7-carboxylate; 5. calcium benzo[1,2,3]thiadiazole-7-carboxylate; 10 to 50 mL of working solution at 80 mg/L containing: 6. choline 3-chlorosalicylate; 7. choline 5-chlorosalicylate; 8. choline 3,5-dichlorosalicylate; 9. sodium 3-chlorosalicylate, 10. potassium 3-chlorosalicylate, 11. calcium 3-chlorosalicylate, 12. magnesium 3-chlorosalicylate four times, at 14-day intervals, with the test product (the first treatment was performed immediately after planting). Control plants were watered with distilled water. During the conducted experiment, plant height was measured before each treatment. Flowering length and plant vigor were also studied. After 20 days from the end of the last treatment, the fresh and dry weight of aboveground parts and roots, and root length were assessed.

### A) Stimulation of Aboveground Plant Parts

TABLE 1
Effect of selected ionic derivatives of aromatic carboxylic
acids on stimulation of tulip plant height and stem diameter.

| | Average plant height [mm] before each treatment | | | | | |
|---|---|---|---|---|---|---|
| | and at the end of the experiment, | | | | | Average stem |
| | Initial | | | | | diameter [mm] at |
| | height | | | | 20 days | the end of the |
| | First | Second | Third | Fourth | after the 4th | experiment |
| Variants of | treat- | treat- | treat- | treat- | treat- | 20 days after the |
| treatment | ment | ment | ment | ment | ment | 4th treatment |
| UTC (untreated | 45.3 a | 101.1 a | 198.2 a | 269.1 a | 400.1 a | 7.2 a |
| control) | | | | | | |
| Plants treated with | 45.7 a | 130.5 b | 257.4 b | 369.1 b | 510.9 b | 8.1 b |
| choline 3- | | | | | | |
| chlorosalicylate | | | | | | |
| 80 mg/L | | | | | | |
| Plants treated with | 46.4 a | 133.6 b | 271.2 b | 375.6 b | 518.0 b | 8.3 b |
| choline | | | | | | |
| benzo[1,2,3]thia- | | | | | | |
| diazole-7- | | | | | | |
| carboxylate | | | | | | |
| 30 mg/L | | | | | | |

Mean values in columns followed by the same letter are not significantly different at p 0,05 according to Duncan's test

The other substances tested 2, 3, 4, 5, 7, 8, 9, 10, 11, 12 also showed stimulating effects ranging from 10-30%. The above results indicate a stimulating effect on plant height and stem diameter of the substances described, especially for choline 3-chlorosalicylate and choline benzo[1,2,3]thiadiazole-7-carboxylate.

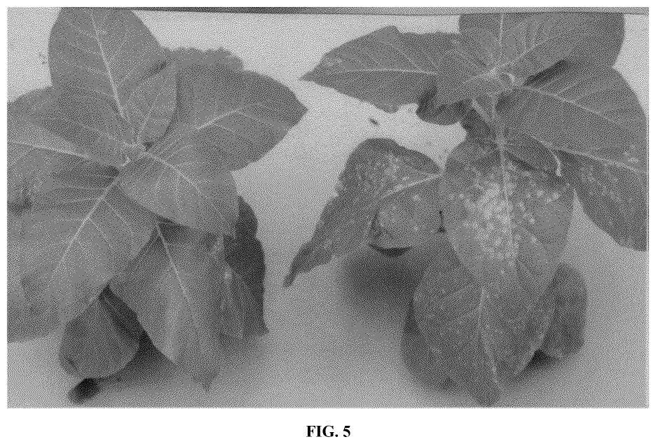

FIG. 4 illustrates the effects of selected ionic derivatives of aromatic carboxylic acids on tulip growth stimulation and plant vigor. On the left side of the photo—UTC (untreated control); in the middle of the photo—plants treated with choline 3-chlorosalicylate; on the right side of the photo—plants treated with choline benzo[1,2,3]thiadiazole-7-carboxylate.

### B) Stimulation of the Growth of the Fresh and Dry Weight of the Aboveground Part of the Tulip Tree

TABLE 2
Effect of selected ionic derivatives of aromatic carboxylic
acids on fresh and dry weight of aboveground parts of tulips.

| | Average fresh | Average dry |
|---|---|---|
| | weight of the | weight of the |
| | above-ground | above-ground |
| Variants of treatment | part [g] | part [g] |
| UTC (untreated control) | 364.7 a | 36.4 a |
| Plants treated with choline 3- | 443.8 b | 41.8 b |
| chlorosalicylate 80 mg/L | | |
| Plants treated with choline | 435.7 b | 44.1 b |
| benzo[1,2,3]thiadiazole-7- | | |
| carboxylate 30 mg/L | | |

Mean values in columns followed by the same letter are not significantly different at p 0,05 according to Duncan's test

The other tested substances 2, 3, 4, 5, 7, 8, 9, 10, 11, 12 also showed a stimulating effect on the growth of fresh and dry weight of the aboveground part of the plant at levels ranging from 8-15%. The above results indicate a stimulating effect on plant height of the described substances, especially choline 3-chlorosalicylate and choline benzo[1,2,3]thiadiazole-7-carboxylate.

### C) Stimulation of Fresh and Dry Root Mass Growth and Root Length

TABLE 3
Effect of selected ionic derivatives of aromatic carboxylic
acids on the fresh and dry weight of tulip bulbs and
on the growth of the root system of tulip bulbs.

| | Average fresh | Average dry | |
|---|---|---|---|
| | weight of the | weight of the | Root |
| | above-ground | above-ground | length |
| Variants of treatment | part [g] | part [g] | [mm] |
| UTC (untreated control) | 44.9 a | 2.8 a | 61.6 a |
| Plants treated with choline 3- | 59.5 b | 5.6 b | 84.3 b |
| chlorosalicylate 80 mg/L | | | |
| Plants treated with choline | 61.0 b | 5.7 b | 84.7 b |
| benzo[1,2,3]thiadiazole-7- | | | |
| carboxylate 30 mg/L | | | |

Mean values in columns followed by the same letter are not significantly different at p 0,05 according to Duncan's test.

The other tested substances 2, 3, 4, 5, 7, 8, 9, 10, 11, 12 also showed a stimulating effect on the growth of fresh and dry root weight of 8-12% and root length of 8-15%. The above results indicate a stimulating effect of the substances described on the growth of fresh and dry root weight, particularly choline 3-chlorosalicylate and choline benzo[1,2,3]thiadiazole-7-carboxylate.

### D) Stimulation of Flowering Length and Overall Plant Vigor

TABLE 4
Effect of selected ionic derivatives of aromatic carboxylic
acids on flowering length and vigor of tulip.

| | | Vigor |
|---|---|---|
| | Flowering | [on a scale |
| Variants of treatment | length [days] | from 1 to 5]*. |
| UTC (untreated control) | 16 | 4.2 a |
| Plants treated with choline 3- | 23 | 4.8 b |
| chlorosalicylate 80 mg/L | | |
| Plants treated with choline | 23 | 4.9 b |
| benzo[1,2,3]thiadiazole-7- | | |
| carboxylate 30 mg/L | | |

*where 1 is a laying plant, 5 is a stiff plant;
Mean values in columns followed by the same letter are not significantly different at p 0.05 according to Duncan's test.

The other tested substances 2, 3, 4, 5, 7, 8, 9, 10, 11, 12 also showed a stimulating effect for an increase in flowering length by 1 to 3 days and plant vigor at the level of 4.3-4.5. The above results indicate that the described substances, especially choline 3-chlorosalicylate and choline benzo[1,2,3]thiadiazole-7-carboxylate, stimulate plant height.

### Example 2. Effect of Selected Ionic Derivatives of Aromatic Carboxylic Acids on Stimulation of Tomato Yield, Content of Health-Promoting Substances in the Yield and Chlorophyll Content in Tomato Leaves after Spraying with the Tested Substances

Tomato seedlings (developmental scale of BBCH 13) were planted in a ground greenhouse. Biostimulant properties were evaluated for the following stimulants: (1) used at a concentration of 30 mg/L and applied by spraying: 1. choline benzo[1,2,3]thiadiazole-7-carboxylate; 2. 2-hydroxyethyl(triethyl)ammonium benzo[1,2,3]thiadiazole-7-carboxylate; 3. 2-hydroxyethyl(tributyl)ammonium benzo[1,2,3]thiadiazole-7-carboxylate; 4. 2-hydroxyethyl(tridodecyl)ammonium benzo[1,2,3]thiadiazole-7-carboxylate; 5. 2-hydroxyethyl(tridodecyl)ammonium benzo[1,2,3]thiadiazole-7-carboxylate; and (2) for the following biostimulants used at a concentration of 80 mg/L and applied by spraying: 6. choline 3-chlorosalicylate; 7. 2-hydroxyethyl(triethyl)ammonium 3-chlorosalicylate; 8. 2-hydroxyethyl(tributyl)ammonium 3-chlorosalicylate; 9. 2-hydroxyethyl(tridecyl)ammonium 3-chlorosalicylate; 10. 2-hydroxyethyl(tridodecyl)ammonium 3-chlorosalicylate. The evaluation of stimulant properties included the influence of tested substances on tomato yield, content of health-promoting substances in yield and chlorophyll content in tomato leaves. For this purpose, tomatoes were sprayed three times at 14-day intervals with the tested substances (spraying was performed with a Solo backpack sprayer). The untreated control plants were sprayed with distilled water. During the experiment, total yield weight, commercial and non-commercial yield and average commercial fruit weight, number of fruits per plant and average fruit weight per plant were evaluated. In addition, fruit division into fractions—caliber (<3.5 cm; >6 cm; 3.5<diameter≥6 cm) was carried out. The experiment was carried out in 4 repetitions with 5 plants in each (a total of 20 plants per combination). Chlorophyll was measured before each treatment and one day after each treatment using Minolta's SPAD-502 chlorophyll meter.

### A) Stimulation of Tomato in Terms of Yield

TABLE 5
Effect of selected ionic derivatives of aromatic carboxylic
acids and a commercially available biostimulator containing
chitosan lactate on the yield of greenhouse tomato.

| | | | Average | Number | Average |
|---|---|---|---|---|---|
| | Total | Commercial | weight of | of fruits | fruit weight |
| | yield | yield | marketable | per | per plant |
| Combination | [kg/m²] | [kg/m²] | fruit [kg/m²] | plant | [g] |
| UTC (untreated | 15.00 a | 14.23 a | 25.57 a | 3.53 a | 121.70 a |
| control) | | | | | |
| Plants treated with | 19.13 b | 18.4 b | 26.25 b | 4.30 b | 154.23 b |
| choline 3- | | | | | |
| chlorosalicylate | | | | | |
| 80 mg/L | | | | | |
| Plants treated with | 18.83 b | 18.13 b | 26.13 b | 4.17 b | 148.70 b |
| choline | | | | | |
| benzo[1,2,3]thiadiazole- | | | | | |
| 7-carboxylate | | | | | |
| 30 mg/L | | | | | |
| Chitosan lactate | 15.30 a | 14.50 a | 25.58 a | 3.53 a | 121.7 a |
| 50 mg/L | | | | | |

Mean values in columns followed by the same letter are not significantly different at p 0,05 according to Duncan's test. Assessment of the significance of differences was performed separately for each parameter studied.

The other tested substances 2, 3, 4, 5, 7, 8, 9 also showed a stimulating effect on parameters describing tomato yield (increase in parameters from 2 to 100). The above results indicate a stimulating effect on plant height of the described substances, especially choline 3-chlorosalicylate and choline benzo[1,2,3]thiadiazole-7-carboxylate.

### B) Stimulation of Biosynthesis of Health-Promoting Substances in Tomato Fruits

TABLE 6
Effect of selected ionic derivatives of aromatic carboxylic acids
and a commercially available biostimulant containing chitosan lactate
on the amount of health-promoting substances in tomato fruits

| | Vit. C | Vit. E | Lycopene | Calcium | Magnesium | °Brix* |
|---|---|---|---|---|---|---|
| Combination | [mg/kg] | [mg/kg] | [mg/kg] | [mg/kg] | [mg/kg] | [g] |
| UTC (untreated control) | 150.0 a | 14.3 a | 25.1 a | 90.9 a | 85.3 a | 5.06 a |
| Plants treated with choline | 191.5 c | 25.6 b | 36.4 b | 97.2 a | 104.5 b | 5.91 b |
| 3-chlorosalicylate | | | | | | |
| 80 mg/L | | | | | | |
| Plants treated with choline | 200.1 c | 25.9 b | 36.5 b | 91.4 a | 106.3 b | 5.92 b |
| benzo[1,2,3]thiadiazole-7- | | | | | | |
| carboxylate 30 mg/L | | | | | | |
| Chitosan lactate 50 mg/L | 165.2 b | 15.3 a | 25.2 a | 102.3 a | 87.1 a | 5.11 a |

*°Brix—sugar content of the liquid expressed in degree of Brix

Mean values in columns followed by the same letter are not significantly different at p 0,05 according to Duncan's test. Assessment of the significance of differences was performed separately for each parameter studied.

The other tested substances 2, 3, 4, 5, 7, 8, 9 also showed a stimulating effect on the content of health-promoting substances in tomato yield (increase in parameters from 4-7%). The above results indicate a stimulating effect on plant height of the described substances, especially choline 3-chlorosalicylate and choline benzo[1,2,3]thiadiazole-7-carboxylate.

### C) Stimulation of Chlorophyll Content in Tomato Leaves

TABLE 7
Effect of selected ionic derivatives of aromatic carboxylic acids and a commercially
available biostimulant containing chitosan lactate on the chlorophyll content
of tomato leaves (measurements were taken with a Minolta SPAD-502 chlorophyll
meter, before each treatment and one day after each treatment).

| | Chlorophyll content/Time of SPAD measurement | | | | | |
|---|---|---|---|---|---|---|
| | | A day | | A day | | A day |
| | | after | | after | | after |
| | Treat- | treat- | Treat- | treat- | Treat- | treat- |
| Combination | ment #1 | ment | ment #2 | ment | ment #3 | ment |
| UTC (untreated control) | 51.3 a | 52.8 a | 50.2 a | 51.3 a | 51.8 a | 50.2 a |
| Plants treated with choline | 50.2 a | 61.4 b | 49.4 a | 60.3 b | 50.9 a | 62.5 b |
| 3-chlorosalicylate | | | | | | |
| 80 mg/L | | | | | | |
| Plants treated with choline | 51.2 a | 63.5 b | 51.4 a | 61.3 b | 51.1 a | 63.2 b |
| benzo[1,2,3]thiadiazole-7- | | | | | | |
| carboxylate 30 mg/L | | | | | | |
| Chitosan lactate 50 mg/L | 51.2 a | 51.1 a | 50.5 a | 49.5 a | 50.7 a | 50.9 a |

Mean values in columns followed by the same letter are not significantly different at p 0,05 according to Duncan's test. Assessment of the significance of differences was performed separately for each parameter studied.

The other tested substances 2, 3, 4, 5, 7, 8, 9 also showed a stimulating effect on chlorophyll content in leaves (increase in parameters from 3 to 6%). The above results indicate a stimulating effect on plant height of the described substances, especially choline 3-chlorosalicylate and choline benzo[1,2,3]thiadiazole-7—carboxylate.

### Example 3. Effect of Selected Ionic Derivatives of Aromatic Carboxylic Acids on Stimulating the Growth of Tomato Seedlings and Fresh and Dry Weight of the Above-Ground Parts of the Plant

Tomato seedlings (12 according to development scale BBCH) were planted into pots with peat substrate (P9 pots having 0.5 l). Plant pots were placed in greenhouse and cultivated for a 1 and a half month. Plants were sprayed with tested substance 3 times, in 10-day interval. Tested substances were 1. choline benzo[1,2,3]thiadiazole-7-carboxylate; 2. 2-methoxyethyl(trimethyl)-ammonium benzo[1,2,3]thiadiazole-7-carboxylate; 3. 2-ethoxyethyl(trimethyl)ammonium benzo[1,2,3]thiadiazole-7-carboxylate; 4. ethyl(trimethyl)ammonium benzo[1,2,3]thiadiazole-7-carboxylate at a concentration of and 30 mg/l and 5. choline 3-chlorosalicylate; 6. 2-methoxyethyl(trimethyl)ammonium 3-chlorosalicylate; 7. 2-ethoxyethyl(trimethyl)ammonium 3-chlorosalicylate; 8. ethyl(trimethyl)ammonium 3-chlorosalicylate at a concentration od 80 mg/l. Plants were sprayed with tested substances three times at 10-day intervals. Plants in peat substrate treated with water only were the control. During the course of the experiment, the height of the plants was measured, and at the end the fresh and dry weight of the aboveground part was evaluated. Moreover chlorophyll content was measured. The experiment was conducted in 4 repetitions with 5 plants in each (a total of 20 plants per each variant of treatment).

### A) Stimulation of Tomato Plant Growth

TABLE 8
Effect of selected ionic derivatives of aromatic
carboxylic acids on tomato plant height [mm].

| | Average plant height [mm] | | | Plant |
|---|---|---|---|---|
| | Initial height, | Before | | height |
| | before first | second | Before third | increase |
| Combination | treatment | treatment | treatment | [mm] |
| Untreated control | 61.2 a | 71.2 a | 151.3 a | 90.1 a |
| Plants treated with choline 3- | 59.3 a | 143.2 b | 345.1 b | 285.8 b |
| chlorosalicylate 80 mg/L | | | | |
| Plants treated with | 60.5 a | 146.7 b | 337.1 b | 276.6 b |
| benzo[1,2,3]thiadiazol-7-choline | | | | |
| carboxylate 30 mg/L | | | | |
| Chitosan lactate [50 mg/l] | 60.1 a | 72.2 a | 159.1 a | 99.0 a |

Mean values in columns followed by the same letter are not significantly different at p 0,05 according to Duncan's test.

The other tested substances 3, 4, 5, also showed a stimulating effect on the growth of pepper plants (increase in parameters from 7-10%). The above results indicate the stimulating effect on plant height of the described substances, especially choline 3-chlorosalicylate and choline benzo[1,2,3]thiadiazole-7-carboxylate.

### B) Effect on Fresh and Dry Mass of the Above-Ground Part of Tomato Plants

TABLE 9
Effect of selected ionic derivatives of aromatic carboxylic
acids and a commercially available biostimulator containing
chitosan lactate on the growth of fresh and dry weight.

| | Average | Average |
|---|---|---|
| | fresh | dry |
| Combination | mass [g] | mass [g] |
| UTC (untreated control) | 184.7 a | 41.1 a |
| Plants treated with choline 3- | 327.7 b | 83.1 b |
| chlorosalicylate 80 mg/L | | |
| Plants treated with benzo[1,2,3]thiadiazol-7- | 333.5 b | 87.5 b |
| choline carboxylate 30 mg/L | | |
| Chitosan lactate | 186.1 a | 42.7 a |

Mean values in columns followed by the same letter are not significantly different at p 0,05 according to Duncan's test. Assessment of the significance of differences was peformed separately for each parameter studied.

The remaining tested substances 2, 3, 4, 6, 7, 8 also showed a stimulating effect on the growth of fresh and dry weight of tomato plants (increase in parameters from 39). The above results indicate the stimulating effect on plant height of the described substances, in particular choline 3-chlorosalicylate and choline benzo[1,2,3]thiadiazole-7-carboxylate.

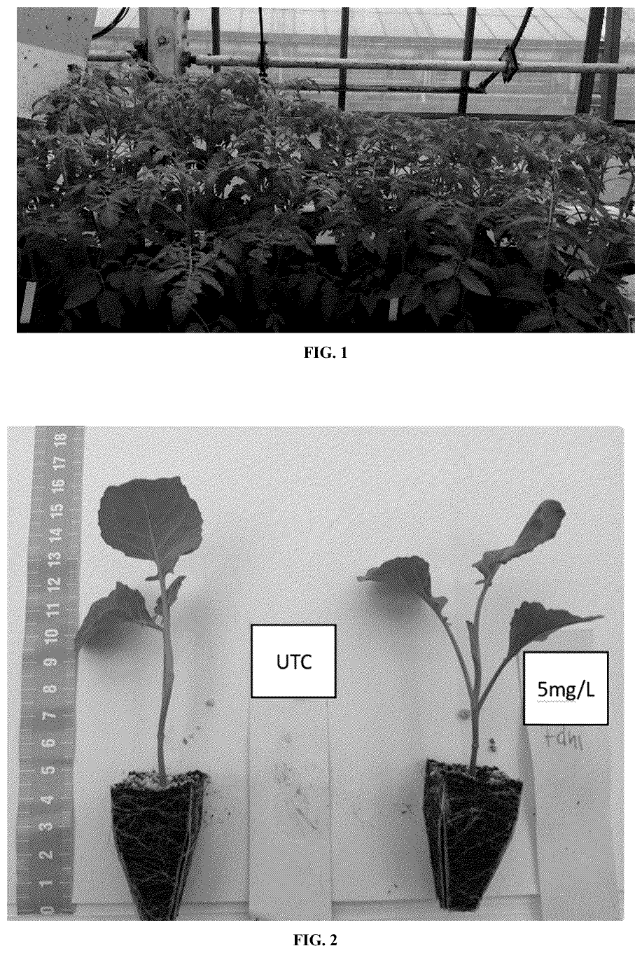

FIG. 1 shows the effects of height stimulation of tomato plants of the Olga variety treated three times with a solution of benzo[1,2,3[thiadiazole-7-carboxylate choline at a concentration of 25 mg/L (left side of the photo) and control plants treated with distilled water (right side of the photo).

### C) Stimulation of Chlorophyll Content in Tomato Leaves

TABLE 10
The influence of selected ionic derivatives of aromatic carboxylic acids and
a commercially available biostimulant containing chitosan lactate on the chlorophyll
content in tomato leaves (reading was taken using a SPAD-502 chlorophyll meter
from Minolta, before the treatment and one day after the treatment).

| | Chlorophyll content/Time of SPAD measurement | | | | | |
|---|---|---|---|---|---|---|
| | | A day | | A day | | A day |
| | | after | | after | | after |
| | Treat- | treat- | Treat- | treat- | Treat- | treat- |
| Combination | ment #1 | ment | ment #2 | ment | ment #3 | ment |
| UTC (untreated control) | 47.1 a | 48.4 a | 50.1 a | 49.3 a | 51.1 a | 46.2 a |
| Plants treated with choline 3- | 48.5 a | 74.6 b | 48.3 a | 76.2 b | 49.5 a | 79.3 b |
| chlorosalicylate 80 mg/L | | | | | | |
| Plants treated with | 49.6 a | 76.1 b | 47.5 a | 81.3 c | 50.1 a | 78.2 b |
| benzo[1,2,3]thiadiazol-7- | | | | | | |
| choline carboxylate 30 mg/L | | | | | | |
| Chitosan lactate [50 mg/l] | 47.6 a | 46.1 a | 47.4 a | 49.4 a | 48.7 a | 48.4 a |

Mean values in columns followed by the same letter are not significantly different at p 0,05 according to Duncan's test. Assessment of the significance of differences was performed separately for each parameter studied.

The other tested substances 2, 3, 4, 6, 7, 8 also showed a stimulating effect on the increase of chlorophyll content in tomato plants (increase in parameters from 5-15%). The above results indicate a stimulating effect on plant height of the described substances, especially choline 3-chlorosalicylate and choline benzo[1,2,3]thiadiazole-7-carboxylate.

### Example 4. The Effect of Selected Ionic Derivatives of Aromatic Carboxylic Acids on Stimulating the Growth of Broccoli Seedlings and Fresh and Dry Weight of the Above-Ground Parts of the Plant

Broccoli seedling were planted into pots with peat substrate (P9 pots having 0.5 l). Pots were placed in greenhouse and cultivated for a 1 month. Tested substances were: 1. choline benzo[1,2,3]thiadiazole-7-carboxylate; 2. 2-fluoroethyl(trimethyl)ammonium benzo[1,2,3]thiadiazole-7-carboxylate; 3. 2-bromoethyl(trimethyl)ammonium benzo[1,2,3]thiadiazole-7-carboxylate; 4. 2-chloroethyl(trimethyl)ammonium benzo[1,2,3]thiadiazole-7-carboxylate; 5. 2-iodoethyl(trimethyl)ammonium benzo[1,2,3]thiadiazole-7-carboxylate used at a concentration of 30 mg/L and 6. 2-hydroxyethyl(trimethyl)ammonium 3-chlorosalicylate (tested also at a concentration of 50 mg/L); 7. 3-chlorosalicylate 2-fluoroethyl(trimethyl)ammonium; 8. 3-chlorosalicylate 2-bromoethyl(trimethyl)ammonium; 9. 3-chlorosalicylate 2-chloroethyl(trimethyl)ammonium; 10. 3-chlorosalicylate 2-iodoethyl(trimethyl)ammonium used at a concentration of 80 mg/L. Plants were sprayed with tested substances three times at 10-day intervals. Plants treated with water only were the untreated control. During the course of the experiment, the height of the plants was measured, and at the end the fresh and dry weight of the aboveground part was evaluated. The experiment was conducted in 4 repetitions with 5 plants in each (a total of 20 plants per each combination).

### A) Stimulation of Broccoli Plant Growth

TABLE 11
Effect of selected ionic derivatives of aromatic
carboxylic acids on broccoli plant height [mm].

| | Average plant height [mm] | | | Plant |
|---|---|---|---|---|
| | Initial height, | | | height |
| | before first | Second | Third | increase |
| Combination | treatment | treatment | treatment | [mm] |
| UTC (untreated control) | 39.4 c | 145.2 a | 179.7 a | 140.3 a |
| Plants treated with choline 3-chlorosalicylate | 37.6 b | 146.4 a | 178.5 a | 140.9 a |
| 50 mg/L | | | | |
| Plants treated with choline 3-chlorosalicylate | 36.5 a | 146.0 a | 179.5 a | 143.0 a |
| 80 mg/L | | | | |
| Plants treated with benzo[1,2,3]thiadiazol-7- | 39.2 c | 187.2 b | 215.7 b | 176.5 b |
| choline carboxylate 30 mg/L | | | | |

Mean values in columns followed by the same letter are not significantly different at p 0,05 according to Duncan's test. Assessment of the significance of differences was performed separately for each parameter studied.

The other tested substances 2, 3, 4, 5, 7, 8, 9, 10 also showed a stimulating effect on the growth of broccoli plants (increase in parameters from 4-15%). The above results indicate a stimulating effect on plant height of the described substances, especially choline 3-chlorosalicylate and choline benzo[1,2,3]thiadiazole-7-carboxylate.

### B) Effect on Fresh and Dry Mass of the Above-Ground Part of Broccoli Plants

TABLE 12
Effect of selected ionic derivatives of aromatic carboxylic
acids on fresh and dry mass of broccoli plant.

| | Average | Average |
|---|---|---|
| | fresh | dry |
| Combination | mass [g] | mass [g] |
| UTC (untreated control) | 5.8 a | 0.4 a |
| Plants treated with choline | 6.8 b | 0.6 b |
| 3-chlorosalicylate 50 mg/L | | |
| Plants treated with choline | 6.7 b | 0.6 b |
| 3-chlorosalicylate 80 mg/L | | |
| Plants treated with benzo[1,2,3]thiadiazol- | 7.8 c | 0.7 b |
| 7-choline carboxylate 30 mg/L | | |

Mean values in columns followed by the same letter are not significantly different at p 0,05 according to Duncan's test. Assessment of the significance of differences was performed separately for each parameter studied.

The other tested substances 2, 3, 4, 5, 7, 8, 9, 10 also showed a stimulating effect on fresh and dry weight of broccoli plants (increase in parameters from 3-10%). The above results indicate a stimulating effect on fresh and dry mass of the described substances, especially choline 3-chlorosalicylate and choline benzo[1,2,3]thiadiazole-7-carboxylate.

FIG. 2 illustrates the stimulation of growth and development of broccoli plants treated once with a solution of choline benzo[1,2,3[thiadiazole-7-carboxylate at a concentration of 5 mg/L (right side of photo) in comparison with control plants treated with distilled water (left side of photo). The plant on the left developed 3 leaves, while the plant treated with the stimulant developed 4—at the same time it moved into the next phase according to the BBCH developmental scale.

### Example 5. Effect of Selected Ionic Derivatives of Aromatic Carboxylic Acids on Stimulating the Growth of Bell Pepper Seedlings and Fresh and Dry Weight of the Above-Ground Parts of the Plant

Pepper seedlings were planted into pots with peat substrate (P9 pots having 0,5 l). Pots were placed in greenhouse and cultivated for a 1 month. Tested substances (by spraying) were 1. benzo[1,2,3]thiadiazole-7-carboxylate choline at a concentrations of 20 mg/l and 30 mg/l and 2. choline 3-chlorosalicylate (tested also at concentration of 50 mg/L); 3. 2-methoxyethyl(trimethyl)ammonium 3-chlorosalicylate; 4. 2-ethoxyethyl(trimethyl)-ammonium 3-chlorosalicylate; 5. ethyl(trimethyl)ammonium 3-chlorosalicylate at a concentration of 80 mg/L. Plants treated with water only were the untreated control. During the course of the experiment, the height of the plants was measured, and at the end the fresh and dry weight of the aboveground part was evaluated. The experiment was conducted in 4 repetitions with 5 plants in each (a total of 20 plants per each variant of treatment).

### A) Stimulation of Bell Pepper Plant Growth

TABLE 13
Effect of selected ionic derivatives of aromatic
carboxylic acids on bell pepper plant height [mm].

| | Average plant height [mm] | | | Plant |
|---|---|---|---|---|
| | Initial height, | | | height |
| | before first | Second | Third | increase |
| Combination | treatment | treatment | treatment | [mm] |
| UTC (untreated control) | 47.5 c | 151.7 b | 185.6 a | 138.1 a |
| Plants treated with choline 3- | 41.5 a | 161.8 c | 221.5 b | 180.0 b |
| chlorosalicylate 80 mg/L | | | | |
| Plants treated with choline | 43.5 b | 150.8 b | 226.6 c | 183.1 c |
| benzo[1,2,3]thiadiazole-7-carboxylate | | | | |
| 20 mg/L | | | | |
| Plants treated with choline | 39.9 a | 142.9 a | 270.8 d | 230.9 d |
| benzo[1,2,3]thiadiazole-7-carboxylate | | | | |
| 30 mg/L | | | | |

Mean values in columns followed by the same letter are not significantly different at p 0,05 according to Duncan's test. Assessment of the significance of differences was performed separately for each parameter studied.

The other tested substances 3, 4, 5, also showed a stimulating effect on the growth of pepper plants (increase in parameters from 7-10%). The above results indicate the stimulating effect on plant height of the described substances, especially choline 3-chlorosalicylate and choline benzo[1,2,3]thiadiazole-7-carboxylate.

### B) Stimulation of Increase of Fresh and Dry Mass of the Above-Ground Part of Pepper Plants

TABLE 14
Effect of selected ionic derivatives of aromatic carboxylic
acids on fresh and dry mass of pepper plant (g).

| | Average | Average |
|---|---|---|
| | fresh | dry |
| Combination | mass [g] | mass [g] |
| UTC (untreated control) | 82.7 a | 11.3a |
| Plants treated with choline | 85.7 b | 12.2 b |
| 3-chlorosalicylate 80 mg/L | | |
| Plants treated with choline | 94.6 c | 13.3 c |
| benzo[1,2,3]thiadiazole-7-carboxylate 20 mg/L | | |
| Plants treated with choline | 115.5 d | 15.6 d |
| benzo[1,2,3]thiadiazole-7-carboxylate 30 mg/L | | |

Mean values in columns followed by the same letter are not significantly different at p 0,05 according to Duncan's test. Assessment of the significance of differences was performed separately for each parameter studied.

The other tested substances 3, 4, 5, also showed a stimulating effect on the growth of fresh and dry weight of bell pepper plants (increase in parameters from 5-12%). The above results indicate a stimulating effect on plant height of the described substances, especially choline 3-chlorosalicylate and choline benzo[1,2,3]thiadiazole-7-carboxylate.

### Example 6. Effect of Selected Ionic Derivatives of Aromatic Carboxylic Acids on Yield Stimulation of Cucumber Cultivated in Field Conditions

Seeds of the field cucumber variety Sremski were sown in the experimental field. The experiment was established in a randomized block design in 4 repetitions of 2 rows of plants each. Cucumbers were sprayed several times at 10-day intervals with the tested product (in different variants of treatment that are indicated below). The control consisted of plants sprayed with distilled water. During the experiment, the effect of choline 3-chlorosalicylate was evaluated. The results obtained were statistically processed by analysis of variance. Duncan's test was used to determine differences between the averages, with a significance level of p=0.05.
1—Untreated control
2—Plants sprayed 2 times with choline 3-chlorosalicylate at a concentration of 80 mg/L
3—Plants sprayed 4 times with choline 3-chlorosalicylate at a concentration of 80 mg/L
4—Plants sprayed 2 times with the trade name Cabrio® Duo 112 EC from BASF (72.0 g/l dimethomorph, 40.0 g/l pyraclostrobin) at a concentration of 0.3%.
5—Plants sprayed 2 times with choline 3-chlorosalicylate at 80 mg/L and 2 times with Cabrio® Duo 112 EC at 0.3%.
6—Plants sprayed 4 times with choline 3-chlorosalicylate at a concentration of 80 mg/L and 2 times with Cabrio® Duo 112 EC at a concentration of 0.3%
7—Plants sprayed with fungicide 4 times (2 treatments with Cabrio® Duo 112 EC concentration 0.3% and 2 treatments with Infinito 687.5 SC from Bayer [propamocarb hydrochloride 625 g/l, fluopicolide 62.5 g/l], products were applied alternately)
8—Plants sprayed 2 times with choline 3-chlorosalicylate at a concentration of 80 mg/L, then sprayed 4 times with fungicide (2 treatments with Cabrio® Duo 112 EC concentration 0.3% and 2 treatments with Infinito 687.5 SC, products were applied alternately)
9—Plants sprayed 4 times with choline 3-chlorosalicylate at a concentration of 80 mg/L, then sprayed 4 times with fungicides (2 treatments with Cabrio® Duo 112 EC, concentration 0.3% and 2 treatments with Infinito 687.5 SC, products were applied alternately)
10—Plants sprayed 4 times with Polyversum® biofungicide marketed by BioAgris

TABLE 15
Effect of tested product on cucumber yield

| Combination number | Cucumber yield [kg/plot] |
|---|---|
| 1 | 15.9 a |
| 2 | 18.8 b |
| 3 | 22.5 c |
| 4 | 22.2 c |
| 5 | 22.8 c |
| 6 | 21.7 c |
| 7 | 24.4 d |
| 8 | 23.8 d |
| 9 | 24.8 d |
| 10 | 18.6 b |

Mean values in columns followed by the same letter are not significantly different at p 0,05 according to Duncan's test.

Use of choline-3-chlorosalicylate in various combinations stimulated the yield of field cucumber.

### Example 7. Effect of Selected Ionic Derivatives of Aromatic Carboxylic Acids on Preventing the Effects of Abiotic Stress on Tomato—Herbicide-Induced Stress

Tomato plants, at the stage of two fully developed leaves, were watered twice with a solution of selected ionic derivatives of aromatic carboxylic acids, at a weekly interval. Tested substances were: choline 3-fluorosalicylate, choline 5-fluorosalicylate, choline 3,5-difluorosalicylate, choline 3-chlorosalicylate, choline 5-chlorosalicylate, choline 3,5-dichlorosalicylate, choline 3-bromosalicylate, choline 5-bromosalicylate, choline 3,5-dibromosalicylate, choline 3-iodosalicylate, choline 5-iodosalicylate, choline 3,5-diiodosalicylate at a concentration of 80 mg/L; and choline benzo(1,2,3)thiadiazole-7-carbosylate and at a concentration of 30 mg/L. Tomato plants watered with water only were the control. One week after the second treatment, the plants were exposed to an herbicide-induced stress (Glyphosate was applied at a dose of 0.005% aqueous solution). 10 days after herbicide application plants not treated with the new derivatives showed a higher degree (20-30%) of herbicide infestation effect, compared to control.

### Example 8. Effect of Selected Ionic Derivatives of Aromatic Carboxylic Acids on Preventing the Effects of Abiotic Stress on Tobacco—Herbicide-Induced Stress

Tobacco plants (Nicotiana tabacum) cv. Xanthi, at the stage of three fully developed leaves, were watered twice with a solution of selected ionic derivatives of aromatic carboxylic acids, at a weekly interval. Tested substances were: choline 2-ethoxy-3-chlorobenzoate, choline 2-methoxy-3-chlorobenzoate, choline 2-acetoxy-3-chlorobenzoate, choline 2-ethoxybenzoate, choline 2-methoxybenzoate, choline 2-acetoxybenzoate, choline 3-chlorosalicylate, choline 5-chlorosalicylate, choline 3,5-dichlorosalicylate at a concentration of 80 mg/L; and choline benzo(1,2,3)thiadiazole-7-carbosylate at a concentration of 30 mg/L. Tobacco plants watered with water only were the control. One week after the second treatment, the plants were exposed to an herbicide-induced stress (Glyphosate was applied at a dose of 0.005% aqueous solution). 10 days after herbicide application, plants not treated with the new derivatives showed a higher degree (21-26%) of herbicide infestation effect, compared to control.

### Example 9. Effect of Selected Ionic Derivatives of Aromatic Carboxylic Acids on Preventing the Effects of Abiotic Stress on Tomato—Lack of Water

Tomato plants, at the stage of two fully developed leaves, were watered with solution of selected ionic derivatives of aromatic carboxylic acids at weekly intervals. The substances tested were: choline 3-chloro-6-fluorosalicylate, choline 3-chloro-6-bromosalicylate, choline 3-chloro-6-iodosalicylate, choline 3-chlorosalicylate, choline 5-chlorosalicylate, choline 3,5-dichlorosalicylate at a concentration of 80 mg/L, and choline benzo(1,2,3)thiadiazole-7-carbosalicylate at a concentration of 30 mg/L. Tomato plants watered with water only were the control. One week after the second treatment, the plants were exposed to the stress factor of drought stress meaning plants watering was stopped. After 10 days, the total weight of plants not treated with ionic derivatives of aromatic carboxylic acids was 7-10% lower than that of plants treated with tested substances.

### Example 10. Effect of Selected Ionic Derivatives of Aromatic Carboxylic Acids on Preventing the Effects of Abiotic Stress on Tobacco—Lack of Water

Tobacco plants (Nicotiana tabacum) cv. Xanthi at the stage of three developed leaves, were watered with solution of selected ionic derivatives of aromatic carboxylic acids at weekly intervals. The substances tested were: choline 3-chloro-6-fluorosalicylate, choline 3-chloro-6-bromosalicylate, choline 3-chloro-6-iodosalicylate, choline 3-chlorosalicylate, choline 5-chlorosalicylate, choline 3,5-dichlorosalicylate at a concentration of 80 mg/L, and choline benzo(1,2,3)thiadiazole-7-carbosalicylate at a concentration of 30 mg/L. Tobacco plant watered with water only were the control. One week after the second treatment, the plants were exposed to the stress factor of drought stress meaning plants watering was stopped. After 10 days, the total weight of plants not treated with ionic derivatives of aromatic carboxylic acids was 10-15% lower than that of plants treated with tested substances.

### Example 11. Effect of Selected Substances on Radish Seed Germination

Radish seeds were placed in water containing the test substances and in water (control sample). Test substances: at a concentration of 40 mg/L: choline 3-chlorosalicylate, choline 5-chlorosalicylate, choline 3,5-dichlorosalicylate, and at a concentration of 15 mg/L: choline benzo(1,2,3)thiadiazole-7-carbosylate. After 4 days, the weight gain of the sprouts was examined in order to evaluate the beneficial effect of tested substances on germination. As a result of the application of tested substances, the weight of the sprouts increased by 5-10%, which means that the tested substances act as a growth regulator, as they accelerate the process of seed germination.

### Example 12. Effect of Choline 3-Chlorosalicylate on Stimulation of Winter Wheat

The experiment was established using the long strip method, in four repetitions Gross area of each plot was 48 m², while the harvest area was 23.25 m² net. Following variants of treatment were tested:
1. Control—no stimulant application, standard fungicide programme
2. Stimulant application at BBCH 32, standard fungicide programme
3. Stimulant application at BBCH 32, no fungicide application
4. Stimulant application at BBCH 55 and 10-12 days later, standard fungicide programme
5. Stimulant application at BBCH 55 and 10-12 days later
Active substance subjected to study was choline 3-chlorosalicylate, applied as spraying at the concentration of 80 mg/L with the working solution of 400 liters per hectare. In this experiment, standard NPK fertilization was applied, using both bulk and liquid fertilizers.

### A) Fresh and Dry Weight of the Above-Ground Part

On the first sampling date—at the beginning of shooting stage—the most biomass was produced by wheat treated with the tested stimulant regardless of whether a fungicide treatment was applied or not. The fresh weight of the aboveground part of the plants in the sprayed objects was higher than that of the control object by 12% and 17%, respectively, while dry weight was higher by 11% and 16%. On the second sampling date, which fell at the end of heading, wheat fresh and dry matter yields were comparable in all experimental facilities. At the last sampling date, at the stage of late milk, the beneficial effect of stimulant treatment at BBCH 55 development scale was particularly evident, compared to stimulant treatment at BBCH 32, regardless of whether a fungicide treatment was applied or not. Wheat treated with the stimulant at BBCH 55 accumulated on average 12% more biomass compared to plants sprayed at an earlier stage, and 31% more than plants of control.

### B) Fresh and Dry Root Mass

On the first sampling date—at the beginning of shooting stage—both fresh and dry root mass in the objects sprayed with the stimulant were higher compared to the control object by 15% and 19%, respectively. On the second sampling date, fresh and dry root weight of wheat was comparable in all experimental facilities. At the last sampling date, at the stage of late milk, the beneficial effect of spraying with the stimulant at BBCH 55 was particularly evident compared to spraying at BBCH 32, regardless of whether a fungicide treatment was applied or not. Wheat treated with the stimulant at BBCH55 accumulated on average 17% more biomass compared to plants sprayed at an earlier stage, and 25% more than plants of control.

### C) Chlorophyll Content

In the first term, significantly higher SPAD values taken using Minolta's SPAD-502 chlorophyll meter were observed for plants treated with stimulant at BBCH 32, regardless of whether a fungicide treatment was applied or not, comparing to plants of control. In the second term, higher SPAD values were observed in plants treated with stimulant at BBCH 32 without fungicide treatment, comparing to plant treated with stimulant at BBCH 32 and fungicides. In the third term, the chlorophyll content of wheat leaves treated with the stimulant was significantly higher compared to the control object, regardless of the technology adopted—with or without fungicide application.

### D) Grain Yield

Regardless of the BBCH phase at which the stimulant was applied, in a technology with no fungicide protection higher grain yields (from 6 to 14%) were obtained compared to technologies with protection. In addition, there was a significant effect of the tested stimulant on the weight of one thousand grains of wheat (an increase of 7 to 10%).

### E) Utilization of Fertilizer Components (Nitrogen, Phosphorus, Potassium)

In the facility where only stimulator spraying was used in phase 55 according to the BBCH development scale in technology without fungicide protection for wheat, an increase in nitrogen (increase by 16%), potassium (increase by 5%) and phosphorus (increase by 12%) was observed. In the case of using the stimulator with standard fungicide protection, the increase occurred respectively: nitrogen (increase by 14%), potassium (increase by 6%) and phosphorus (increase by 12%). All differences were statistically significant.

In the facility where only stimulator spraying was used in stage 32 according to the BBCH development scale, wheat took up higher amounts of nitrogen (an increase of 7%), potassium (an increase of 8%) and phosphorus (an increase of 9%). In the case of using the stimulator with standard fungicide protection, the increase occurred by: nitrogen (increase by 7%), potassium (increase by 6%) and phosphorus (increase by 8%). All differences were statistically significant.

### F) Utilization of Mineral Nutrients in the Soil (not Supplied to Soil/Plant in Form of a Fertilizer)

In the facility where only stimulator spraying was used in phase 55 according to the BBCH development scale in technology without fungicide protection for wheat, an increase in magnesium (increase by 6%) and calcium (increase by 5%) was observed. In the case of using the stimulator with standard fungicide protection, the increase occurred respectively: magnesium (increase by 5%), calcium (increase by 4%). All differences were statistically significant.

In the facility where only spraying with a stimulator was used in stage 32 according to the BBCH development scale, wheat took up higher amounts of magnesium (an increase of 5%) and calcium (an increase of 6%). In the case of using the stimulator with standard fungicide protection, the increase occurred respectively: magnesium (increase by 4%), calcium (increase by 4%). All differences were statistically significant.

### G) Protein and Gluten Content of Wheat Grain

In the facility where only stimulator spraying was used in phase 55 according to the BBCH development scale in technology without fungicide protection for wheat, an increase in protein content (increase by 10%) and gluten (increase by 8%) was observed. When the stimulator was used with standard fungicide protection, the following increases occurred: proteins (increase by 11%), gluten (increase by 9%). All differences were statistically significant.

In the facility where only stimulator spraying was used in stage 32 according to the BBCH development scale, higher amounts of protein (an increase of 8%) and gluten (an increase of 7%) were observed in the wheat grain. In the case of using the stimulator with standard fungicide protection, the increase occurred by: protein (increase by 10%), gluten (increase by 8%). All differences were statistically significant.

The application of choline 3-chlorosalicylate stimulated the growth of fresh and dry weight of the aboveground and root parts, the increase of chlorophyll content in leaves, yield, thousand-grain weight. In addition, the application of the stimulant spray favorably improved the utilization of bulk and liquid fertilizer components (nitrogen, potassium, phosphorus) and the uptake of minerals from the soil (calcium, magnesium). In addition, the spray increased the protein content of wheat grain. Beneficial effects were observed regardless of the stage according to the BBCH development scale at which the spraying was carried out and the cultivation technology (without/with fungicide protection).

### Example 13. Effect of Choline Benzo(1,2,3)Thiadiazole-7-Carboxylate on Stimulation of Winter Wheat Parameters

The experiment was established using the long strip method, in four repetitions Gross area of each plot was 48 m2, while the harvest area was 23.25 m2 net. Following variants of treatment were tested:
1. Control—no stimulant application, standard fungicide programme,
2. Stimulant application at BBCH 32, standard fungicide programme
3. Stimulant application at BBCH 32, no fungicide application
4. Stimulant application at BBCH 55 and 10-12 days later, standard fungicide programme
5. Stimulant application at BBCH 55 and 10-12 days later
   The stimulant tested (choline benzo(1,2,3)thiadiazole-7-carboxylate) was applied as a spray at a concentration of 30 mg/L per 400 l of water.
   In the experiment, uniform NPK fertilization was applied, using both bulk and liquid fertilizers.

### A) Fresh and Dry Weight of the Above-Ground Part

On the first sampling date—at the beginning of shooting—the most biomass was produced by wheat treated with the tested stimulant regardless of whether or not a fungicide treatment was applied. The fresh weight of the aboveground part of the plants in the sprayed objects was higher than in the control object by 11% and 14%, respectively, while the dry weight was higher by 10% and 14%. On the second sampling date, which fell at the end of heading, wheat fresh and dry matter yields were comparable in all experimental facilities. At the last sampling date, at the stage of late milk, the beneficial effect of spraying the stimulant at stage 55 according to the BBCH developmental scale was particularly evident compared to spraying at stage 32 according to the BBCH developmental scale, regardless of whether a fungicide treatment was applied or not. Wheat treated with the stimulant at stage 55 according to the BBCH development scale accumulated on average 13% more biomass compared to plants sprayed at an earlier stage, and 30% more than the control object.

### B) Fresh and Dry Root Mass

On the first sampling date—at the beginning of shooting—both fresh and dry root mass in the objects sprayed with the stimulant were 12% and 14% higher, respectively, compared to the control object. On the second sampling date, fresh and dry root weight of wheat was comparable in all experimental facilities. At the last sampling date, at the stage of late milk of wheat, the beneficial effect of spraying with the stimulant at stage 55 according to the BBCH development scale was particularly evident compared to spraying at stage 32 according to the BBCH development scale, regardless of whether a fungicide treatment was applied or not. Wheat treated with the stimulant at stage 55 according to the BBCH developmental scale accumulated an average of 14% more biomass compared to plants sprayed at an earlier stage, and 21% compared to the control object.

### C) Chlorophyll Content

In the first term, significantly higher readings using Minolta's SPAD-502 chlorophyll meter relative to the control were observed in facilities with stimulant spray application of wheat at stage 32 according to the BBCH development scale, both in technology with and without fungicide protection. In the second term, the highest readings were obtained in the facility with stimulant application at stage 32 according to the BBCH developmental scale, without fungicide protection. In the third term, the chlorophyll content of wheat leaves treated with the stimulant was significantly higher compared to the control object, regardless of the technology adopted—with or without fungicide application

### D) Grain Yield

Regardless of the growth stage at which the stimulant was applied, the technology without fungicide protection produced higher grain yields (from 10 to 12%) compared to the technology with protection. In addition, there was a significant effect of the tested growth stimulant on the weight of one thousand grains of wheat (an increase of 8 to 10%).

### E) Utilization of Fertilizer Components (Nitrogen, Phosphorus, Potassium)

In the facility where only the stimulant spray was applied at stage 55 according to the BBCH development scale in the technology without fungicide protection, an increase in the uptake of nitrogen (increase of 15%) potassium (increase of 6%) and phosphorus (increase of 11%) was observed. When the stimulant was applied along with standard fungicide protection, the increase was in nitrogen (12% increase), potassium (7% increase) and phosphorus (11% increase), respectively. All differences were statistically significant.

In the facility where only the stimulant spray was applied at stage 32 according to the BBCH development scale, the wheat took up higher amounts of nitrogen (8% increase) potassium (8% increase) and phosphorus (9% increase). When the stimulant was applied along with standard fungicide protection, the increase was in nitrogen (7% increase), potassium (7% increase) and phosphorus (9% increase), respectively. All differences were statistically significant.

### F) Utilization of Mineral Nutrients in the Soil (not Supplied to the Soil/Plant in the Form of Fertilizer)

In the facility where only the stimulant spray was applied at stage 55 according to the BBCH development scale in the technology without fungicide protection, an increase in the uptake of magnesium (10% increase) and calcium (8% increase) was observed. When the stimulant was applied together with standard fungicide protection, the increase was in magnesium (11% increase), calcium (12% increase), respectively. All differences were statistically significant.

In the facility where only the stimulant spray was applied at stage 32 according to the BBCH development scale, the wheat took up higher amounts of magnesium (9% increase) and calcium (7% increase). When the stimulant was applied along with standard fungicide protection, the increase was in magnesium (8% increase), calcium (7% increase), respectively. All differences were statistically significant.

### G) Protein and Gluten Content of Wheat Grain

In the facility where only the stimulant spray was applied at stage 55 according to the BBCH development scale in the technology without fungicide protection, an increase in protein (11% increase) and gluten (9% increase) was observed. When the stimulant was applied along with standard fungicide protection, the increase was in protein (12% increase), gluten (10% increase), respectively. All differences were statistically significant.

In the facility where only the stimulant spray was applied at stage 32 according to the BBCH development scale, higher amounts of protein (11% increase) and gluten (8% increase) were observed in wheat grain. When the stimulant was applied along with standard fungicide protection, the increase was in protein (12% increase), gluten (9% increase), respectively. All differences were statistically significant.

The application of choline 3-chlorosalicylate spray stimulated the growth of fresh and dry weight of the aboveground and root parts, an increase in the chlorophyll content of the leaves, yield, thousand-grain weight. In addition, the application of the stimulant spray favorably improved the utilization of bulk and liquid fertilizer components (nitrogen, potassium, phosphorus) and the uptake of minerals from the soil (calcium, magnesium). In addition, the spray increased the protein content of wheat grain. Beneficial effects were observed regardless of the BBCH stage at which the spray was applied and the cultivation technology (without/fungicide protection).

### Example 14 Effect of Choline 3-Chlorosalicylate on Stimulation of Winter Rapeseed Parameters

The experiment was established using the long strip method in four repetitions. The dimensions of the plots at establishment were 96 m² gross, while the harvestable area was 23.25 m² net.

The following facilities were included in the experiment:
1. control, no stimulant application, standard fungicide protection
2. stimulant applied at stage 31-32 according to the BBCH development scale, standard fungicide protection
3. stimulant applied at stage 53-55 according to the BBCH development scale, without fungicide protection
   The stimulant tested (choline 3-chlorosalicylate) was applied as a spray at a concentration of 80 mg/L per 400 l of water/ha.

In the experiment, uniform NPK fertilization was applied, using both bulk and liquid fertilizers.

### A) Fresh and Dry Weight of the Above-Ground Part

On the first sampling date, plants were treated only with the stimulant, without fungicide application. Under the stimulant spray application at the 31-32 stage according to the BBCH development scale, plants produced 36% (fresh weight) and 34% (dry weight) more biomass, respectively, compared to plants from the control object.

On the second date, plants in the fungicide-controlled facility were previously treated with a fungicide. It was found that regardless of the cultivation technology adopted, rapeseed treated with the stimulant accumulated on average 28% more fresh weight and 32% more dry weight compared to the control.

At the last harvest date, which fell at the stage of fully ripe, rapeseed in the control facility had the lowest biomass. In the facility where the stimulant was applied in technology with fungicide protection, plants accumulated on average 38% more aboveground dry matter compared to plants in the control facility, and in the facility where only the tested stimulant was applied—by as much as 42%.

### B) Fresh and Dry Root Mass

At each sampling date, rapeseed produced significantly more root mass compared to the control, both in the facility with fungicide protection (from 10% to 23%) and without protection (from 8% to 22%).

### C) Chlorophyll Content

On the first measurement date, no differences were observed between measurements of chlorophyll content in the tested objects. On the second and third measurement dates, oilseed rape treated with the stimulant, regardless of the combination—with a fungicide (more by 5% n the first date and more by 7% n the second date) or without a fungicide (more by 6% n the first date and more by 5% n the second date), accumulated significantly more chlorophyll in the leaves compared to plants in the control object, as evidenced by higher values of readings with the Minolta SPAD-502 chlorophyll meter.

### D) Rapeseed Yield

Regardless of the cultivation technology adopted, spraying with the tested stimulant had a beneficial effect on rapeseed yields. In the technology without fungicide protection, seed yields obtained in the facility where plants were treated with the stimulant at the 31-32 stage according to the BBCH development scale were 10% higher compared to the control. The highest yields were achieved when both fungicide and stimulant treatments were included in the rapeseed crop (20%). There was an effect of the stimulant tested on the weight of one thousand seeds of rapeseed, which was higher in the tested combinations by 10% compared to the control.

### E) Utilization of Fertilizer Components (Nitrogen, Phosphorus, Potassium)

In the facility where only the stimulant spray was applied at stage 55 according to the BBCH development scale in the technology without fungicide protection, an increase in the uptake of nitrogen (11% increase) potassium (7% increase) and phosphorus (10% increase) was observed. When the stimulant was applied along with standard fungicide protection, the increase was in nitrogen (11% increase), potassium (8% increase) and phosphorus (10% increase), respectively. All differences were statistically significant.

In the facility where only the stimulant spray was applied at stage 32 according to the BBCH development scale, the rapeseed took up higher amounts of nitrogen (9% increase) potassium (8% increase) and phosphorus (7% increase). When the stimulant was applied along with standard fungicide protection, the increase was in nitrogen (6% increase), potassium (6% increase) and phosphorus (7% increase), respectively. All differences were statistically significant.

### F) Utilization of Mineral Nutrients in the Soil (not Supplied to the Soil/Plant in the Form of Fertilizer)

In the facility where only the stimulant spray was applied at stage 55 according to the BBCH development scale in the technology without fungicide protection, an increase in the uptake of magnesium (increase of 12%) and calcium (increase of 10%) was observed. When the stimulant was applied together with standard fungicide protection, the increase was in magnesium (10% increase), calcium (9% increase), respectively. All differences were statistically significant.

In the facility where only the stimulant spray was applied at stage 32 according to the BBCH development scale, the rapeseed took up higher amounts of magnesium (12% increase) and calcium (8% increase). When the stimulant was applied along with standard fungicide protection, the increase was in magnesium (4% increase), calcium (5% increase), respectively. All differences were statistically significant.

### G) Fat Content

In the facility where only the stimulant spray was applied at stage 55 according to the BBCH development scale in the technology without fungicide protection, an increase in fat content (5% increase) was observed. When the stimulant was applied together with standard fungicide protection, an increase of 6% in fat content occurred. All differences were statistically significant.

In the facility where only the stimulant spray was applied at stage 32 according to the BBCH development scale, higher fat contents of 8% without fungicide and 6% with fungicide were observed. All differences were statistically significant.

The application of choline 3-chlorosalicylate spray stimulated the growth of fresh and dry weight of the aboveground and root parts, an increase in the chlorophyll content of leaves, yield, weight of one thousand grains. In addition, the application of the stimulant spray favorably improved the utilization of bulk and liquid fertilizer components (nitrogen, potassium, phosphorus) and the uptake of minerals from the soil (calcium, magnesium). In addition, spraying increased the fat content of rapeseed. Beneficial effects were observed regardless of the BBCH stage at which spraying was carried out and the cultivation technology (without/fungicide protection).

### Example 15. Effect of Benzo(1,2,3)Thiadiazole-7-Carboxylate Choline on Stimulation of Winter Rapeseed Parameters

The experiment was established using the long strip method in four repetitions. The dimensions of the plots at establishment were 96 m² gross, while the harvestable area was 23.25 m² net.

The following facilities were included in the experiment:
1. control, no stimulant application, standard fungicide protection
2. stimulant applied at stage 31-32 according to the BBCH development scale, standard fungicide protection
3. stimulant applied at stage 53-55 according to the BBCH development scale, no fungicide protection

The stimulant tested (choline benzo(1,2,3)thiadiazole-7-carboxylate) was applied as a spray at a concentration of 25 mg/L per 400 l of water/ha.

In the experiment, uniform NPK fertilization was applied, using both bulk and liquid fertilizers.

### A) Fresh and Dry Weight of the Above-Ground Part

On the first sampling date, plants were treated only with the stimulant, without fungicide application. Under the stimulant spray application at the 31-32 stage according to the BBCH development scale, plants produced 28% (fresh weight) and 29% (dry weight) more biomass, respectively, compared to plants from the control object.

On the second date, plants in the fungicide-controlled facility were previously treated with a fungicide. It was found that regardless of the cultivation technology adopted, rapeseed treated with the stimulant accumulated on average 24% more fresh weight and 26% more dry weight compared to the control.

At the last harvest date, which fell at the stage of fully ripe, rapeseed in the control facility had the lowest biomass. In the facility where the stimulant was applied in technology with fungicide protection, plants accumulated on average 38% more aboveground dry matter compared to plants in the control facility, and in the facility where only the tested stimulant was applied—by as much as 32%.

### B) Fresh and Dry Root Mass

At each sampling date, rapeseed produced significantly more root mass compared to the control, both in the facility with fungicide protection (8% to 16%) and without protection (4% to 14%).

### C) Chlorophyll Content

On the first measurement date, no differences were observed between measurements of chlorophyll content in the studied objects. On the second and third measurement dates, oilseed rape treated with the stimulant, regardless of the combination—with a fungicide (more by 6% n the first date and more by 8% n the second date) or without a fungicide (more by 8% n the first date and more by 12% n the second date), accumulated significantly more chlorophyll in the leaves compared to plants in the control object, as evidenced by higher values of readings with the Minolta SPAD-502 chlorophyll meter.

### D) Rapeseed Yield

Regardless of the cultivation technology adopted, spraying with the tested stimulant had a beneficial effect on rapeseed yields. In the technology without fungicide protection, seed yields obtained in the facility where plants were treated with the stimulant at the 31-32 stage according to the BBCH development scale were 9% higher compared to the control. The highest yields were achieved when both fungicide and stimulant treatments were included in the rapeseed crop (12%). There was an effect of the stimulant tested on the thousand-seed weight of rapeseed, which was higher in the tested combinations by 12% compared to the control.

### E) Utilization of Fertilizer Components (Nitrogen, Phosphorus, Potassium)

In the facility where only the stimulant spray was applied at stage 55 according to the BBCH development scale in the technology without fungicide protection, an increase in the uptake of nitrogen (increase of 12%) potassium (increase of 8%) and phosphorus (increase of 9%) was observed. When the stimulant was applied along with standard fungicide protection, the increase was in nitrogen (9% increase), potassium (8% increase) and phosphorus (11% increase), respectively. All differences were statistically significant.

In the facility where only the stimulant spray was applied at stage 32 according to the BBCH development scale, the rapeseed took up higher amounts of nitrogen (8% increase), potassium (7% increase) and phosphorus (9% increase). When the stimulant was applied along with standard fungicide protection, the increase was in nitrogen (5% increase), potassium (6% increase) and phosphorus (5% increase), respectively. All differences were statistically significant.

### F) Utilization of Mineral Nutrients in the Soil (not Supplied to the Soil/Plant in the Form of Fertilizer)

In the facility where only the stimulant spray was applied at stage 55 according to the BBCH development scale in the technology without fungicide protection, an increase in the uptake of magnesium (13% increase) and calcium (9% increase) was observed. When the stimulant was applied together with standard fungicide protection, the increase was in magnesium (9% increase), calcium (10% increase), respectively. All differences were statistically significant.

In the facility where only the stimulant spray was applied at stage 32 according to the BBCH development scale, the rapeseed took up higher amounts of magnesium (11% increase) and calcium (9% increase). When the stimulant was applied along with standard fungicide protection, the increase was in magnesium (5% increase), calcium (5% increase), respectively. All differences were statistically significant.

### G) Fat Content

An increase in fat content (% increase) was observed in the facility where only the stimulant spray was applied at stage 55 according to the BBCH development scale in the technology without fungicide protection. When the stimulant was applied together with standard fungicide protection, an increase of 6% in fat content occurred. All differences were statistically significant.

In the facility where only the stimulant spray was applied at stage 32 according to the BBCH development scale, higher fat contents of 8% without fungicide and 6% with fungicide were observed. All differences were statistically significant.

The application of benzo(1,2,3)thiadiazole-7-carboxylate choline spray stimulated the growth of fresh and dry weight of the aboveground and root parts, the increase of chlorophyll content in leaves, yield, thousand-grain weight. In addition, the application of the stimulant spray favorably improved the utilization of bulk and liquid fertilizer components (nitrogen, potassium, phosphorus) and the uptake of minerals from the soil (calcium, magnesium). In addition, the spray increased the fat content of rapeseed. Beneficial effects were observed regardless of the BBCH stage at which spraying was carried out and the cultivation technology (without/with fungicide protection).

### Example 16. Effect of Selected Ionic Derivatives of Aromatic Carboxylic Acids on Growth Stimulation of Lettuce Cultivated Under Normal and Drought Stress Condition

The purpose of the study was to determine the effect of foliar spraying with test solutions on lettuce (Lactuca sativa L. ‘Zeralda’) plants. The plants were sprayed 7 and 14 days after transplanting to a permanent site. Substances used at a concentration of 100 mg/L, choline 3-chlorosalicylate, and at a concentration of 50 mg/L: choline benzo(1,2,3)thiadiazole-7-carbosylate. After another 7 days, drought stress was induced (plants in the selected combinations were not watered until symptoms of leaf wilting appeared), followed by proper watering of the plants again. Control plants were watered in turn to a constant weight. After 3 days after re-watering, chlorophyll fluorescence was measured (OJIP test, methodology described, among others, in Kalaji H. M., 2011. Impact of abiotic stress factors on chlorophyll fluorescence in plants of selected barley cultivars Hordeum vulgare L. SGGW Publishing House, Warsaw [https://badanieroslin.pl/wp-content/uploads/2018/11/Oddzialywanie-abiotycznych-czynnikow. . . ..pdf]) and the condition of the plants was assessed.

### A) Plant Yields

There was a positive effect of the application of the preparation applied 1-time on improving the yield of aboveground parts of plants grown under the standard water regime (Table 16). In turn, under stress conditions, the application of the preparation (1-time application) significantly improved the yield of plants. Similar correlations applied to the weight of the whole plant including the root system, which was highest with the 1-time application of the preparation. The formulation had a positive effect on the yield of plants under drought stress.

TABLE 16
head weight and head weight including root system.

| | Head weight (g/plant) | | | Head and root weight (g/plant) | | |
|---|---|---|---|---|---|---|
| Plant spraying | Watered | Drought | Average | Watered | Drought | Average |
| UTC (untraced control) | 53.3 b | 45.2 a | 49.3 a | 743.5 abc | 64.2 a | 69.6 a |
| Choline 3-chlorosalicylate | 50.6 ab | 52.8 b | 51.3 a | 73.2 abc | 71.7 abc | 72.5 a |
| 100 mg/L; double | | | | | | |
| application | | | | | | |
| choline | 65.3 c | 62.5 c | 65.1 b | 85.2 c | 84.4 bc | 84.6 b |
| benzo(1,2,3)thiadiazole-7- | | | | | | |
| carbosilane 50 mg/L; single | | | | | | |
| application | | | | | | |
| choline | 51.6 b | 51.6 ab | 53.4 a | 69.2 a | 70.6 ab | 69.4 a |
| benzo(1,2,3)thiadiazole-7- | | | | | | |
| carbosylate 50 mg/L; | | | | | | |
| double application | | | | | | |

### B) Relative Water Content (RWC) and Leaf Dry Matter Content.

A significant positive effect of the application on the value of the RWC index in leaves was shown for plants grown under a standard water regime.

TABLE 17
relative water content (RWC) and leaf dry matter content (%).

| | RWC (%) | | | % dry matter | | |
|---|---|---|---|---|---|---|
| Spraying plants | Watered | Drought | Average | Watered | Drought | Average |
| Control (distilled water) | 73.1 ab | 62.2 a | 66.5 a | 3.00 a | 2.81 a | 2.91 a |
| Choline 3- | 73.2 ab | 71.9 ab | 73.3 a | 3.96 b | 3.55 b | 3.72 b |
| chlorosalicylate 100 | | | | | | |
| mg/L; double | | | | | | |
| application | | | | | | |
| choline | 77.1 b | 71.5 ab | 74.5 a | 3.51 b | 3.59 b | 3.75 b |
| benzo(1,2,3)thiadiazole- | | | | | | |
| 7-carbosylate 50 mg/L; | | | | | | |
| single application | | | | | | |
| choline | 74.3 b | 64.0 a | 75.0 a | 5.73 c | 3.61 a | 4.67 b |
| benzo(1,2,3)thiadiazole- | | | | | | |
| 7-carbosylate 50 mg/L; | | | | | | |
| double application | | | | | | |

Lea dry matter content was significantly lowest for the control sprayed distilled water—and significantly highest for plants in the standard water regime for the formulation applied 2 times.

### C) Photosynthetic Parameters

The measurement was carried out after inducing drought stress, followed by re-watering the plants to a constant weight. The application of formulation I (choline 3-chlorosalicylate) and II (choline benzo(1,2,3)thiadiazole-7-carboxylate) (for 2-fold application), in the case of drought-stressed plants modified (outlined trend) the value of initial fluorescence (FO)—with respect to control plants, it was significantly higher for plants sprayed with formulation 11. In the case of maximum fluorescence (FM), the value of the index was at a similar level regardless of the tested combination (for drought stress)—and at the same time the evaluated parameter was significantly higher for control plants after treatment with formulation 11—indicating the response of electron acceptors in photosystem mL (PSII). In control plants, the fluorescence index (Fv) was significantly higher after treatment with formulation os, and in plants subjected to drought stress for formulation I and double application of formulation 11—a positive indication of PSII performance. This parameter is lowered by stresses. FM/FO was at similar levels in control plants sprayed with distilled water, as well as in all combinations subjected to drought stress, which may indicate that there was no destruction of photosystem 11. For Fv/FO, trends were similar in control and drought-stressed plants. Significant differences in Fv/FM were observed for single and double applications of formulation 11 in drought-stressed plants.

TABLE 18
Effect of the studied factors on the rate of energy flow (a) and specific energy
flows through the RC reaction center (b) at the third measurement date.

| | | | | | | |
|---|---|---|---|---|---|---|
| Spraying plants | F0 | FM | Fv | FM/F0 | Fv/F0 | Fv/FM |

Control

| | | | | | | |
|---|---|---|---|---|---|---|
| Control (distilled water) | 4985 a | 32652 a | 27667 a | 6.55 abc | 5.55 abc | 0.85 bc |
| Choline 3-chlorosalicylate 100 | 4877 a | 34278 a | 29401 a | 7.32 c | 6.32 c | 0.86 c |
| mg/L; double application | | | | | | |
| choline benzo(1,2,3)thiadiazole-7- | 6762 b | 41874 b | 35112 b | 6.19 abc | 5.19 abc | 0.84 abc |
| carbosylate 50 mg/L; single | | | | | | |
| application | | | | | | |
| choline benzo(1,2,3)thiadiazole-7- | 7100 b | 45265 b | 38164 b | 6.52 ab | 5.52 abc | 0.85 abc |
| carbosylate 50 mg/L; two | | | | | | |
| applications. | | | | | | |

Drought

| | | | | | | |
|---|---|---|---|---|---|---|
| Control (distilled water) | 5075 a | 30862 a | 25787 a | 6.09 ab | 5.09 ab | 0.84 abc |
| Choline 3-chlorosalicylate 100 | 5524 a | 33908 a | 28384 b | 6.16 ab | 5.16 ab | 0.84 ab |
| mg/L; double application | | | | | | |
| choline benzo(1,2,3)thiadiazole-7- | 5675 a | 32147 a | 26472 a | 5.67 a | 4.67 a | 0.83 a |
| carbosylate 50 mg/L; single | | | | | | |
| application | | | | | | |
| choline benzo(1,2,3)thiadiazole-7- | 4965 a | 34715 a | 29750 b | 6.99 bc | 5.99 ab | 0.86 c |
| carbosylate 50 mg/L; two | | | | | | |
| applications. | | | | | | |

| | | | | | |
|---|---|---|---|---|---|
| Spraying plants | Pi_Abs* | ABS/RC*. | TRo/RC* | ETo/RC* | DIo/RC* |

Control

| | | | | | |
|---|---|---|---|---|---|
| Control (distilled water) | 9.97 c | 1.10 a | 1.30 a | 0.77 a | 0.20 a |
| Choline 3-chlorosalicylate 100 | 12.74 d | 1.48 b | 1.72 b | 1.08 b | 0.24 a |
| mg/L; double application | | | | | |
| choline | 4.47 a | 1.45 b | 1.74 b | 0.87 ab | 0.28 b |
| benzo(1,2,3)thiadiazole-7- | | | | | |
| carbosylate 50 mg/L; single | | | | | |
| application | | | | | |
| choline | 10.33 ab | 1.46 b | 1.74 b | 0.93 ab | 0.28 b |
| benzo(1,2,3)thiadiazole-7- | | | | | |
| carbosylate 50 mg/L; double | | | | | |
| application | | | | | |

Drought

| | | | | | |
|---|---|---|---|---|---|
| Control (distilled water) | 9.07 c | 1.08 a | 1.28 a | 0.74 a | 0.22 a |
| Choline 3-chlorosalicylate 100 | 7.77 b | 1.20 a | 1.44 a | 0.81 a | 0.24 a |
| mg/L; double application | | | | | |
| choline | 7.47 b | 1.12 a | 1.36 a | 0.75 a | 0.24 a |
| benzo(1,2,3)thiadiazole-7- | | | | | |
| carbosylate 50 mg/L; single | | | | | |
| application | | | | | |
| choline | 12.61 d | 1.14 a | 1.34 a | 0.84 ab | 0.20 a |
| benzo(1,2,3)thiadiazole-7- | | | | | |
| carbosylate 50 mg/L; double | | | | | |
| application | | | | | |

* explanation of abbreviations:
Pi_Abs—PSII functioning index calculated on the basis of energy absorption
ABS/RC—flux of photons absorbed by chlorophyll molecules per reaction center (RC)
TRo/RC—flow of photons retained (reducing QA) in PSII per reaction center
ETo/RC—electron flow behind the QA per RC
DIo/RC—thermal dissipation of excitation energy behind QA per RC
QA—plastoquinone bound to D2 protein

The study showed significant differences in energy flows, with different energy flows for some parameters in control and drought-stressed plants. Pi_Abs was different in analogous combinations in control and stressed plants, while the flow of absorbed energy through one active reaction center (ABS/RC) increased after treatment in control plants—it was at the same time similar to each other (an unproven tendency) in drought-stressed plants. A similar trend was observed for the flow of retained photons (reducing QA) in PSII per reaction center (TRo/RC). The flow of electrons downstream of QA per RC (ETo/RC) showed an increasing trend—in many cases not statistically proven, however, only in the case of preparation I in relation to the control. Thermal dissipation of excitation energy per RC was at similar levels in stressed plants, while significantly higher after treatment with formulation II in the case of control.

### Example 17. Effect of Choline 3-Chlorosalicylate and Choline Benzo(1,2,3)Thiadiazole-7-Carboxylate on Stimulation of Shoot Growth and the Number of Flowers in Apple Trees

The experiment was conducted in a randomized block design, in four repetitions, with 4 apple plants in each repetition (in the form of M9 rootstocks having around 50 cm that were transplanted into 2 L pots). In the spring, after leaf formation, total of 4 sprays (20 ml per plant, concentration of choline 3-chlorosalicylate was equal to 100 mg/L and choline benzo(1,2,3)thiadiazole-7-carboxylate was equal to 25 mg/L) were made at an interval of 14 days. Flowers were counted during the experiment, and the number of shoots and leaves in the fall.

TABLE 19
Average number of shoots, flowers and leaves per plant.

| | Average | Average | Average |
|---|---|---|---|
| | number of | number of | number of |
| | shoots | flowers | leaves per |
| | per plant | per plant | plant |
| Untreated control | 3.2 a | 22.1 a | 36.9 a |
| Choline 3- | 6.7 b | 30.3 b | 45.3 b |
| chlorosalicylate | | | |
| 100 mg/L; | | | |
| Choline | 6.5 b | 31.1 b | 47.1 b |
| benzo(1,2,3)thiadiazole- | | | |
| 7-carbosylate 25 mg/L; | | | |

The results indicate that choline 3-chlorosalicylate and choline benzo(1,2,3)thiadiazole-7-carboxylate have a stimulation effect on parameters such as flower number, shoot number, and leaf number in apple plants (M9 rootstocks)

### Example 18. Effect of Selected Ionic Derivatives of Aromatic Acids on Stimulation of Yield Parameters, Sugar Content, Sodium, Potassium and Nitrogen Content in Sugar Beet Pulp

The experiment was conducted in a randomized block design, in four repetitions. The five-replicate experimental plots had an area of 22.5 m² (10 mn long and 2.25 mn wide). Applications of crop protection products and test substances were made with a wheelbarrow sprayer, designed for spraying the experimental plots. The amount of spray was 400 liters per hectare, the concentration of stimulants 2-4 was 80 mg/L while 5 was at a concentration of 25 mg/L. The experiment involved the use of 5 tested substances, each in 3 different variants, and an untreated control with the tested biostimulants. Treatments were carried out according to the following scheme:

TABLE 20
Scheme of treatments in the study of the effect of ionic derivatives of
aromatic acids on stimulating the growth of yield, sugar content, sodium
content, potassium content and nitrogen content in sugar beet pulp.

| Treatment | | | | | |
|---|---|---|---|---|---|
| term | I | II | III | IV | V |
| Untreated | | | | | |
| control | | | | | |
| Variant A | Biostimulant | Biostimulant | | Biostimulant | Biostimulant |
| Variant B | Biostimulant | Biostimulant | Fungicide | | Biostimulant |
| Variant B | Biostimulant | Biostimulant | Fungicide | Biostimulant | Biostimulant |

TABLE 21
Results of stimulation of yield parameters, sugar content,
sodium, potassium and nitrogen content in sugar beet pulp

| | | | Biological | K content | Na content | Nα content | |
|---|---|---|---|---|---|---|---|
| | | Root | sugar | [mmol/ | [mmol/ | [mmol/ | Technologic |
| | | yield | content | 1000 g | 1000 g | 1000 g | sugar |
| | Combination | [t/ha]. | [%]. | pulp]. | pulp]. | pulp]. | yield [t/ha]. |
| 1 | Untreated Control | 62.79 a | 19.74 a | 38.20 a | 5.05 ab | 8.50 c | 11.26 a |
| 2A | 4 × 3ClSA | 68.11 b | 19.60 a | 38.58 a | 4.60 a | 6.95 b | 12.15 ab |
| 2B | 2 × 3ClSA + | 70.08 b | 20.01 a | 37.88 a | 5.35 b | 6.33 ab | 12.80 b |
| | fungicide + | | | | | | |
| | 1 × 3ClSA | | | | | | |
| 2C | 2 × 3ClSA + | 68.16 b | 19.61 a | 38.60 a | 5.03 ab | 6.88 b | 12.16 ab |
| | fungicide + | | | | | | |
| | 2 × 3ClSA | | | | | | |
| 3A | 4 × 5ClSA | 68.13 b | 20.01 a | 38.33 a | 5.30 b | 8.15 b | 12.40 a |
| 3B | 2 × 5ClSA + | 61.61 a | 19.86 a | 36.28 a | 5.05 b | 6.58 b | 11.16 a |
| | fungicide + | | | | | | |
| | 1 × 5ClSA | | | | | | |
| 3C | 2 × 5ClSA + | 65.53 ab | 19.85 a | 36.80 a | 4.95 ab | 5.63 a | 11.88 a |
| | fungicide + | | | | | | |
| | 2 × 5ClSA | | | | | | |
| 4A | 4 × 3.5ClSA | 61.56 a | 19.72 a | 37.83 a | 4.75 a | 6.18 ab | 11.06 a |
| 4B | 2 × 3.5ClSA + | 66.30 ab | 19.49 a | 38.20 a | 4.60 a | 5.73 a | 11.78 a |
| | fungicyd + | | | | | | |
| | 1 × 3.5ClSA | | | | | | |
| 4C | 2 × 3.5ClSA + | 65.38 ab | 19.53 a | 37.55 a | 4.98 ab | 6.20 ab | 11.64 a |
| | fungicide + | | | | | | |
| | 2 × 3.5ClSA | | | | | | |
| 5A | 4 × [Chol][BTHCOO] | 67.76 b | 20.01 a | 37.88 a | 5.35 b | 6.33 ab | 12.80 a |
| 5B | 2 × Chol][BTHCOO] + | 66.54 ab | 19.61 a | 38.60 a | 5.03 ab | 6.88 b | 12.16 ab |
| | fungicide + | | | | | | |
| | 1 × Chol][BTHCOO] | | | | | | |
| 5C | 2 × [Chol][BTHCOO] + | 65.86 ab | 20.01 a | 38.33 a | 5.30 b | 8.15 b | 12.40 a |
| | fungicide + | | | | | | |
| | 2 × [Chol][BTHCOO] | | | | | | |

3ClSA: 3-chlorosalicylate choline;
5ClSA: 5-chlorosalicylate of choline;
3,5ClSA: 3,5-chlorosalicylate of choline,
[Chol][BTHCOO]: choline benzo(1,2,3)thiadiazole-7-carboxylate

The abovementioned results indicate that the use of stimulants, regardless of the number of applications and their use with or without fungicide treatment, result in increase of parameters such as: root yield, biological sugar content, yield of technological sugar. In turn, a decrease in the content of potassium, sodium and nitrogen in the pulp is observed (or their content at a similar level as in the untreated control), which is a positive result, taking into account the requirements of sugar mills to keep these parameters as low as possible. Particularly preferred results were obtained for combinations where sprays were applied with a stimulant containing sodium benzo(1,2,3)thiadiazole-7-carboxylate and sodium 3-chlorosalicylate.

### Example 19. Effect of Selected Ionic Derivatives of Aromatic Acids on Preventing the Effects of Abiotic Stress of Low Temperature

The purpose of the study was to determine the effect of foliar spraying with test solutions on lettuce plants (Lactuca sativa L. ‘Zeralda’) under abiotic stress conditions—low temperature. After 7 and 14 days after transplanting the plants to a permanent place, spraying of the plants was carried out. Substances used at a concentration of 100 mg/L, choline 3-chlorosalicylate, and at a concentration of 50 mg/L: choline benzo(1,2,3)thiadiazole-7-carbosylate. After another 7 days, low temperature stress was induced—plants were grown in three glass chambers with a day temperature of 22° C. and night temperatures of 4, 12 and 20° C. until harvest. Yield results are shown in Table 22.

TABLE 22
Head weight and head weight including root system.

| | Head weight (g/plant) | | |
|---|---|---|---|
| Spraying plants | 4° C. | 12° C. | 20° C. |
| UTC (untreated control) | 32.6 a | 45.2 a | 55.5 a |
| Choline 3-chlorosalicylate | 48.9 b | 52.8 b | 65.3 b |
| 100 mg/L; double application | | | |
| choline benzo(1,2,3)thiadiazole-7- | 47.6 b | 54.9 b c | 65.1 b |
| carbosylate 50 mg/L; single application | | | |

The above results indicate that the studied stimulants have a beneficial effect on preventing the effects of abiotic stress—low temperature.

### Example 20. Effect of Choline 3-Chlorosalicylate on the Growth and Development of Poinsettia Plant Under Drought Stress Conditions

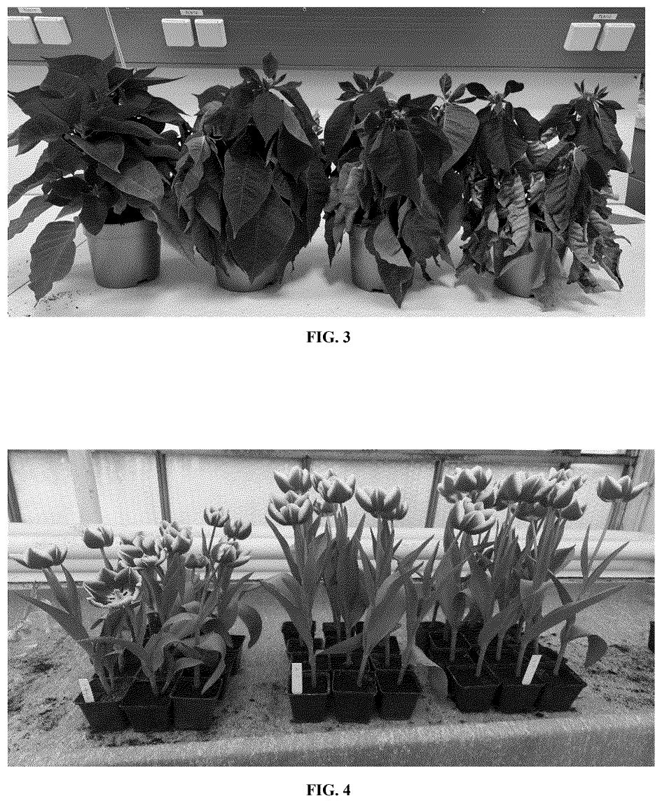

Plants of poinsettia were treated three times with choline 3-chlorosalicylate at a concentration of 50 mg/L, while control plants were treated with distilled water. The plants were then subjected to drought stress—they were not watered for 7 days. FIG. 3 shows a photo of the plants tested after 7 days: the two plants on the left side of the photo are plants treated with choline 3-chlorosalicylate solution, while the two plants on the right side of the photo are control plants. The plants treated with the stimulant retained their vigor; no drought effects were observed, while the control plants wilted.

### Example 21: No Direct Effect on Tobacco Mosaic Virus (TMV) Infectivity of Selected Ionic Aromatic Derivatives of Carboxylic Acids

Purified tobacco mosaic virus, (TMV) at a concentration of approximately 3 μg/ml was mixed with following substances: 1. choline 3-chlorosalicylate; 2. choline 5-chlorosalicylate; 3. choline 3,5-dichlorosalicylate; 4. Sodium 3-chlorosalicylate; 5. Potassium 3-chlorosalicylate; 6. Calcium 3-chlorosalicylate; 7. Magnesium 3-chlorosalicylate (concentration 80 mg/L), in a 1:1 ratio and incubated for 30 min at room temperature. The control variant consisted of TMV incubated in water. All virus suspensions were used to mechanically infect Xanthi tobacco leaves, whose reaction to infection is manifested in easily quantifiable local necrotic spots (hypersensitivity, local infection).

After 5 days, the area of leaf covered by necrotic spots compared to total leaf area was calculated for plants of all variants of treatment. The results indicate differences between the tested combinations of 0-5 percent. which allow to conclude that the tested substances do not directly affect TMV infectivity (tested substances have no antiviral effect).

TABLE 23
Degree of infection of the tobacco leaves with TMV-
containing solution observed 5 days infection

| | Substance | % of necrotic spots |
|---|---|---|
| | Control (virus without tested substance) | 100 |
| 1. | choline 3-chlorosalicylate; | 98 |
| 2. | Choline 5-chlorosalicylate; | 100 |
| 3. | Choline 3,5-dichlorosalicylate; | 95 |
| 4. | Sodium 3-chlorosalicylate, | 97 |
| 5. | Potassium 3-chlorosalicylate, | 95 |
| 6. | calcium 3-chlorosalicylate | 100 |

### Example 22: Selected Ionic Aromatic Derivatives of Carboxylic Acids have No Direct Effect on Bacterial Infectivity (Pseudomonas syringae pv. Tomato)

Proceeding as in example 21, Pseudomonas syringae pv. Tomato bacteria were tested and incubated in bacterial medium.

After 2 days, the concentration of bacteria on the plants was compared, and differences of <5% were determined, which allows us to conclude that the test substances do not directly affect the infectivity of bacteria.

TABLE 24
Degree of infection of the tobacco plant with the
bacterium (Pseudomonas syringae pv. tomato).
After 5 days of infecting the plant with a solution
containing the bacteria and the test substance.

| | Substance | % of necrotic spots |
|---|---|---|
| | Virus without added test substance (control) | 100 |
| 1. | choline 3-chlorosalicylate; | 99 |
| 2. | Choline 5-chlorosalicylate; | 98 |
| 3. | Choline 3,5-dichlorosalicylate; | 100 |
| 4. | Sodium 3-chlorosalicylate, | 95 |
| 5. | Potassium 3-chlorosalicylate, | 98 |
| 6. | calcium 3-chlorosalicylate | 97 |

### Example 23: Selected Ionic Aromatic Derivatives of Carboxylic Acids have No Direct Effect on Fungal Infectivity (Powdery Mildew)

Proceeding as in Example 21, the test was performed on the powdery mildew fungus, which was then incubated on the medium.

After 2 days, the results of the test and control were compared, and differences of <40 were determined, which allows us to conclude that the preparation does not directly affect fungal infectivity.

TABLE 25
Degree of infection of the tobacco plant with the fungus
- powdery mildew 5 days after infecting the plant with
a solution containing the fungus and the test substance.

| | Substance | % of necrotic spots |
|---|---|---|
| | Virus without added substance (control) | 100 |
| 1. | choline 3-chlorosalicylate; | 100 |
| 2. | Choline 5-chlorosalicylate; | 98 |
| 3. | Choline 3,5-dichlorosalicylate; | 96 |
| 4. | Sodium 3-chlorosalicylate, | 96 |
| 5. | Potassium 3-chlorosalicylate, | 98 |
| 6. | calcium 3-chlorosalicylate | 97 |

### Example 24: Selected Ionic Aromatic Derivatives of Carboxylic Acids Applied to Plant Roots (Watering) are Very Effective in Protecting Against Biotic Stress Caused by Viral Infection

Tobacco plants (Nicotiana tabacum) cv. Xanthi at the stage of three developed leaves, were watered twice with a solution of substances 1. choline 3-chlorosalicylate; 2. choline 5-chlorosalicylate; 3. 3,5-dichlorosalicylate of choline; 4. 3-chlorosalicylate of sodium, 5. 3-chlorosalicylate of potassium, 6. 3-chlorosalicylate of calcium, 7. 3-chlorosalicylate of magnesium at a concentration of 10 or 40 or 80 mg/L, at one-week intervals. The control was tobacco plants watered only with water. One week after the second treatment of the plants with the preparation, mechanical infection of the leaves with Tobacco Mosaic Virus (TMV) was carried out. This consisted of rubbing carborundum-sprinkled leaves several times with fingers soaked in a suspension of purified virus at a concentration of about 2 μg/ml. In order to evaluate the effectiveness of the tested substances in protection against biotic stresses, the TMV model and tobacco cv. Xanthi characterized by an interaction in the form of hypersensitivity phenomenon, i.e. the formation of easily quantifiable necrotic spots, were used. A comparison of the number of spots on the leaves of control and treated plants shows that the application of the preparation to the roots of tobacco plants completely reduces the impact of the biotic factor—viral infection—on the plant, as shown in FIG. 5, showing the reduction of necrotic spots (effectiveness) on the tobacco plant 7 days after infection with TMV (Tobacco Mosaic Virus). The tobacco plant was watered with a solution containing 80 mg/L choline 3-chlorosalicylate before infection.

TABLE 26
Degree of reduction of necrotic spots (effectiveness)
on the leaf of the tobacco plant with TMV (Tobacco
Mosaic Virus) 7 days after infection of the plants.

| | | % reduction in necrotic | | |
|---|---|---|---|---|
| | | spots compared to Control | | |
| | Substance | 10 mg/L | 40 mg/L | 80 mg/L |
| | Control (watering with water) | 0 | | |
| 1. | choline 3-chlorosalicylate; | 80 | 100 | 100 |
| 2. | choline 5-chlorosalicylate; | 34 | 62 | 98 |
| 3. | choline 3,5-dichlorosalicylate; | 36 | 58 | 96 |
| 4. | sodium 3-chlorosalicylate, | 24 | 63 | 95 |
| 5. | potassium 3-chlorosalicylate, | 32 | 65 | 98 |
| 6. | calcium 3-chlorosalicylate | 33 | 65 | 97 |

### Example 25: Selected Ionic Aromatic Derivatives of Carboxylic Acids Applied by Spraying Leaves Very Effectively Prevent the Occurrence of Biotic Stress Caused by Viral Infection

Proceeded as in Example 24, with plants immunized by spraying twice at weekly intervals, with a solution of the substances 1. choline 3-chlorosalicylate; 2. 5-chlorosalicylate of choline; 3. 3,5-dichlorosalicylate of choline; 4. 3-chlorosalicylate of sodium, 5. 3-chlorosalicylate of potassium, 6. 3-chlorosalicylate of calcium, 7. 3-chlorosalicylate of magnesium at concentrations of 10, 40, 80 mg/L. The substances tested effectively protected the treated leaves from biotic stress caused by TMV infection at concentrations of 40 and 80 mg/L. The other substances, depending on the concentration used, showed efficacy ranging from 20-85%.

TABLE 27
Degree of reduction of necrotic spots (effectiveness)
on the leaf of the tobacco plant with TMV (Tobacco
Mosaic Virus) 7 days after infection of the plants.

| | | % reduction in necrotic | | |
|---|---|---|---|---|
| | | spots compared to control | | |
| | Substance | 10 mg/L | 40 mg/L | 80 mg/L |
| | Control (watering with water) | 0 | | |
| 1. | choline 3-chlorosalicylate; | 80 | 100 | 85 |
| 2. | choline 5-chlorosalicylate; | 39 | 61 | 74 |
| 3. | choline 3,5-dichlorosalicylate; | 36 | 47 | 54 |
| 4. | sodium 3-chlorosalicylate, | 24 | 29 | 55 |
| 5. | potassium 3-chlorosalicylate, | >10 | >10 | 15 |
| 6. | calcium 3-chlorosalicylate | 20 | 30 | 36 |

### Example 26: Selected Ionic Aromatic Derivatives of Carboxylic Acids are More Effective in Preventing the Occurrence of Biotic Stress Caused by Viral Infection than the Comparison Material, the Commercially Available Formulation BION™

Tobacco plants (Nicotiana tabacum) cv. Xanthi at the stage of three developed leaves were sprayed once with solutions of substance 1-5 or BION™ at concentrations of 10, 40, 80 mg/L. A week later, they were mechanically infected with TMV by rubbing the leaves with a square soaked in a suspension of purified virus, with a concentration of about 2 μg/ml. The level of protection against the effects of biotic stress was evaluated by comparing the number of necrotic spots caused by TMV on the leaves of plants treated with substances 1-5 and BION™ and control plants. The study showed that even at a concentration of 40 mg/L, substance 1 was more effective in preventing the occurrence of biotic stress.

Table 28 shows the amount of necrotic spots resulting from virus infection on plants exposed to substances 1-5 and BION™ compared to the control. A reduction in the number of necrotic spots indicates protection from the effects of the biotic agent on the plant.

TABLE 28
Degree of reduction of necrotic spots (effectiveness)
on the leaf of the tobacco plant caused by viral
infection with comparison material - BION.

| | | % reduction in | | |
|---|---|---|---|---|
| | | necrotic spots | | |
| | | compared to control | | |
| | | 10 | 40 | 80 |
| | Substance | mg/L | mg/L | mg/L |
| | Control | 0 | | |
| | BION (commercial product) | 65 | 89 | 89 |
| 1. | choline 3-chlorosalicylate; | 58 | 92 | 98 |
| 2. | 2-hydroxyethyl(triethyl)ammonium | 15 | 26 | 37 |
| | 3-chlorosalicylate; | | | |
| 3. | 2-hydroxyethyl(tributyl)ammonium | 26 | 26 | 30 |
| | 3-chlorosalicylate | | | |
| 4. | 2-hydroxyethyl(tridecyl)-ammonium | 25 | 46 | 65 |
| | 3-chlorosalicylate; | | | |
| 5. | 2-hydroxyethyl(tridodecyl)ammonium | 59 | 64 | 89 |
| | 3-chlorosalicylate | | | |

### Example 27: Protection Against Biotic Stress Remains Highly Effective Even 21 Days after the Last Application of Selected Ionic Derivatives of Aromatic Carboxylic Acids and Occurs at Successive Leaf Levels of Treated Plants

They proceeded as in Example 24, using substances 1-4 at concentrations of 10, 40 and 80 mg/L for treatment, with treated and control plants divided into 3 batches and their leaves infected with the virus 1, 2 and 3 weeks after the last treatment, respectively. The results showed that protection against biotic stress following TMV infection was fully effective 3 weeks after the last watering of the plants with the preparations occurred on the 6th-7th consecutive leaf. A similar effect occurred with spraying.

TABLE 29
Degree of reduction of necrotic spots (effectiveness)
on the leaf of a tobacco plant infected with TMV 3
weeks after the last treatment with substances 1-4.

| | | % reduction | | |
|---|---|---|---|---|
| | | in necrotic spots | | |
| | | compared to control | | |
| | | 10 | 40 | 80 |
| | Substance | mg/L | mg/L | mg/L |
| | Control | 0 | | |
| 1. | choline 3-chlorosalicylate; | 58 | 92 | 98 |
| 2. | 2-methoxyethyl(trimethyl)ammonium | 15 | 26 | 37 |
| | 3-chlorosalicylate | | | |
| 3. | 2-ethoxyethyl-(trimethyl)ammonium | 26 | 26 | 30 |
| | 3-chlorosalicylate | | | |
| 4. | ethyl(trimethyl)ammonium 3-chlorosalicylate; | 25 | 46 | 65 |

### Example 28: Protection Against Biotic Stress in Winter Barley Following Brome Mosaic Virus (BMV) Infection in Oats

Barley plants in 10 cm diameter pots were watered twice, at weekly intervals, with 70 ml of substance 1-17 solution at a concentration of 80 mg/L. The control was barley plants watered with water. The following week, after the second watering of the plants with the test substance, one young developed leaf was sprinkled with carborundum to obtain small wounds through which the virus was introduced when the leaf was mechanically infected with a finger dipped in a purified BMV suspension, at a concentration of about 10 μg/L. Two weeks later, it was determined from the disease symptoms that, compared to the control, all treated barley plants did not show the effects of biotic stress.

TABLE 30
Degree of reduction of necrotic spots (effectiveness) on leaves
of BMV-infected barley plants 2 weeks after infection.

| | | % reduction |
|---|---|---|
| | | in necrotic spots |
| | Substance | compared to control |
| | Control | 0 |
| 1. | choline 3-chlorosalicylate | 100 |
| 2. | 2-hydroxyethyl(trimethyl)ammonium | 98 |
| | 3-chlorosalicylate | |
| 3. | 2-fluoroethyl(trimethyl)ammonium | 56 |
| | 3-chlorosalicylate; | |
| 4. | 2-bromoethyl(trimethyl)ammonium | 95 |
| | 3-chlorosalicylate; | |
| 5. | 2-chloroethyl(trimethyl)ammonium | 36 |
| | 3-chlorosalicylate; | |
| 6. | 2-iodoethyl(trimethyl)ammonium | 64 |
| | 3-chlorosalicylate | |
| 7. | choline 3-fluorosalicylate, | 56 |
| 8. | choline 5-fluorosalicylate | 88 |
| 9. | choline 3,5-difluorosalicylate | 27 |
| 10. | choline 5-chlorosalicylate | 36 |
| 11. | choline 3,5-dichlorosalicylate | 77 |
| 12. | choline 3-bromosalicylate, | 21 |
| 13. | choline 5-bromosalicylate, | 69 |
| 14. | choline 3,5-dibromosalicylate | 38 |
| 15. | choline 3-iodosalicylate | 77 |
| 16. | choline 5-iodosalicylate | 35 |
| 17. | choline 3,5-diiodosalicylate | 46 |

### Example 29: Effect of Choline 3-Chlorosalicylate Concentration on Biotic Stress Protection Efficiency in Barley

They proceeded as in Example 28, with the plants treated (watered) with a choline 3-chlorosalicylate solution of 10 mg/L. 3-chlorosalicylate also at a lower concentration prevented biotic stress following BMV infection at 69%.

TABLE 31
Degree of reduction of necrotic spots (effectiveness) on leaves
of BMV-infected barley plants 2 weeks after infection.

| | % reduction in necrotic |
|---|---|
| Substance | spots compared to control |
| Control | 0 |
| choline 3-chlorosalicylate | 69 |

### Example 30: Protection Against Biotic Stress on Tobacco (Nicotiana tabacum) Cv. Xanthi Following Infection by the Bacterium Pseudomonas syringae pv. Tomato, by Watering the Plants

Tobacco plants at the stage of 3 developed leaves were watered twice, at a weekly interval, with a solution of test substances 1-11 at concentrations of 10, 40, 80 mg/L. The control was tobacco plants watered with water only. One week after the second treatment, a suspension of Pst bacteria at a concentration of 10⁵ was spotted into the leaves using an insulin syringe (without a needle). The suspension was prepared from a two-day culture of Pst on solid medium. Protection against biotic stress was evaluated by the proliferation of bacteria in the leaves at the insertion site and the formation of a necrotic spot, compared to the control. As a result of the application of substance 1-11, the establishment of bacterial infection and the formation of necrotic spots similar to those observed in the control were not observed on treated plants.

TABLE 32
Degree of reduction of necrotic spots (effectiveness) on leaves of
tobacco plants infected with Pst bacterium 2 weeks after infection.

| | | % reduction | | |
|---|---|---|---|---|
| | | in necrotic spots | | |
| | | compared to control | | |
| | | 10 | 40 | 80 |
| | Substance | mg/L | mg/L | mg/L |
| | Control | 0 | | |
| 1. | choline 3-chlorosalicylate | 65 | 72 | 89 |
| 2. | choline 2-ethoxy-3-chlorobenzoate | 24 | 33 | 39 |
| 3. | choline 2-methoxy-3-chlorobenzoate | >10 | 25 | 39 |
| 4. | choline 2-acetoxy-3-chlorobenzoate | >10 | 28 | 35 |
| 5. | choline 2-ethoxybenzoate | 56 | 60 | 82 |
| 6. | choline 2-methoxybenzoate | 54 | 68 | 67 |
| 7. | choline 2-acetoxybenzoate | 25 | 29 | 69 |
| 8. | choline 5-chlorosalicylate | 28 | 36 | 80 |
| 9. | choline 3,5-dichlorosalicylate | 27 | 38 | 84 |
| 10. | 3-chloro-6-fluorosalicylate choline, | 19 | 28 | 47 |
| 11. | choline 3-chloro-6-bromosalicylate | 24 | 29 | 40 |

### Example 31: Effectiveness of Preventing Biotic Stress by Choline 3-Chlorosalicylate at Elevated Bacterial Cell Concentrations of Pseudomonas syringae pv. Tomato

We proceeded as in Example 30, with the effectiveness of protection against biotic stress tested against bacterial cell concentrations raised to about 10⁶. The Pst bacterium in plants treated with choline 3-chlorosalicylate at a concentration of 80 mg/L undertook a slight activity but the effects of infection were inhibited and no local necrotic spots formed as a characteristic effect of bacterial proliferation.

TABLE 33
Degree of reduction of necrotic spots (effectiveness) on leaves of
tobacco plants infected with Pst bacterium 2 weeks after infection.

| | | % reduction in necrotic |
|---|---|---|
| | Substance | spots compared to control |
| | Control | 0 |
| 1. | choline 3-chlorosalicylate; | 85 |

### Example 32: Protection Against Biotic Stress in Tomato Following Infection by the Bacterium Pseudomonas syringae pv. Tomato, by Watering the Plants

Tomato plants, at the stage of the first pair of developed true leaves, were watered twice with a solution of substance 1-6 at a concentration of 80 mg/L, at a weekly interval. The control was tomato plants watered only with water. One week after the second treatment, a suspension of Pst bacteria at a concentration of 10⁵ was spot-injected into the leaves using an insulin syringe (without a needle). The suspension was prepared from a two-day culture of Pst on solid medium. Protection against biotic stress was evaluated by the proliferation of bacteria in the leaves at the insertion site and the formation of a necrotic spot, compared to the control. As a result of the application of substances 1-6, the establishment of bacterial infection and the formation of necrotic spots similar to those observed in the control were not observed on treated plants.

TABLE 34
Degree of reduction of necrotic spots (effectiveness) on leaves of
tomato plants infected with Pst bacterium 2 weeks after infection.

| | | % reduction in necrotic |
|---|---|---|
| | Substance | spots compared to control |
| | Control | 0 |
| 1. | choline 3-chlorosalicylate; | 92 |
| 2. | choline 5-chlorosalicylate; | 87 |
| 3. | choline 3,5-dichlorosalicylate; | 59 |
| 4. | sodium 3-chlorosalicylate, | 53 |
| 5. | potassium 3-chlorosalicylate, | 87 |
| 6. | calcium 3-chlorosalicylate | 86 |

### Example 33: Protection Against Biotic Stress in Lycopersicon Esculentum Mill Tomato Following Infection by the Bacterium Pseudomonas Syringae pv. Tomato, by Spraying the Plants

The procedure was followed as in Example 32, with the plants sprayed. Substances 1-6 at a concentration of 80 mg/L applied by spraying twice prevents biotic stress, fully protecting the tomato from the effects of bacterial infection.

TABLE 35
Degree of reduction of necrotic spots (effectiveness) on leaves of
tomato plants infected with Pst bacterium 2 weeks after infection.

| | | % reduction in necrotic |
|---|---|---|
| | Substance | spots compared to control |
| | Control | 0 |
| 1. | choline 3-chlorosalicylate; | 93 |
| 2. | choline 5-chlorosalicylate; | 88 |
| 3. | choline 3,5-dichlorosalicylate; | 57 |
| 4. | sodium 3-chlorosalicylate, | 51 |
| 5. | potassium 3-chlorosalicylate, | 84 |
| 6. | calcium 3-chlorosalicylate | 82 |

### Example 34: Protection Against Biotic Stress in Tomato Following Fungal Infection of Powdery Mildew by Watering the Plants

Tomato plants, at the stage of the first pair of developed true leaves, were watered twice with a solution of substance 1-32 at a concentration of 80 mg/L, at a weekly interval. The control was tomato plants watered only with water. One week after the second treatment, a suspension of the powdery mildew fungus was spot-injected into the leaves using an insulin syringe (without a needle). The suspension was prepared from culture on solid medium. Protection against biotic stress was evaluated by comparing the area of leaves affected by infection. As a result of the application of substances 1-32 on treated plants, the establishment of fungal infection (up to 95%) and the formation of infected areas similar to those observed in the control were not observed.

TABLE 36
Degree of reduction of necrotic spots (effectiveness) on leaves of tomato
plants infected with powdery mildew fungus 2 weeks after infection.

| | | % reduction in necrotic |
|---|---|---|
| | Substance | spots compared to control |
| | Control | 0 |
| 1. | choline 3-chlorosalicylate; | 95 |
| 2. | choline 5-chlorosalicylate; | 90 |
| 3. | choline 3,5-dichlorosalicylate; | 85 |
| 4. | sodium 3-chlorosalicylate, | 88 |
| 5. | potassium 3-chlorosalicylate, | 84 |
| 6. | calcium 3-chlorosalicylate | 69 |
| 7. | 2-hydroxyethyl(triethyl)ammonium 3-chlorosalicylate; | 58 |
| 8. | 2-hydroxyethyl(tributyl)ammonium 3-chlorosalicylate; | 66 |
| 9. | 2-hydroxyethyl(tridecyl)-ammonium 3-chlorosalicylate; | 64 |
| 10. | 2-hydroxyethyl(tridodecyl)ammonium 3-chlorosalicylate | 47 |
| 11. | 2-methoxyethyl(trimethyl)ammonium 3-chlorosalicylate; | 67 |
| 12. | ethyl(trimethyl)ammonium 3-chlorosalicylate | 63 |
| 13. | choline 3-fluorosalicylate | 52 |
| 14. | choline 5-fluorosalicylate, | 54 |
| 15. | choline 3,5-difluorosalicylate, | 70 |
| 16. | choline 3-bromosalicylate, | 72 |
| 17. | choline 5-bromosalicylate, | 79 |
| 18. | choline 3,5-dibromosalicylate, | 72 |
| 19. | choline 3-iodosalicylate, | 69 |
| 20. | choline 5-iodosalicylate, | 65 |
| 21. | choline 3,5-diiodosalicylate | 78 |
| 22. | 2-fluoroethyl(trimethyl)ammonium 3-chlorosalicylate; | 36 |
| 23. | 2-bromoethyl(trimethyl)ammonium 3-chlorosalicylate; | 54 |
| 24. | 2-chloroethyl(trimethyl)ammonium 3-chlorosalicylate; | 57 |
| 25. | 2-iodoethyl(trimethyl)ammonium 3-chlorosalicylate | 47 |
| 26. | 2-ethoxyethyl-(trimethyl)ammonium 3-chlorosalicylate; | 55 |
| 27. | choline 2-ethoxy-3-chlorobenzoate, | 43 |
| 28. | choline 2-methoxy-3-chlorobenzoate, | 49 |
| 29. | choline 2-acetoxy-3-chlorobenzoate, | 45 |
| 30. | choline 2-ethoxybenzoate, | 43 |
| 31. | choline 2-methoxybenzoate, | 44 |
| 32. | choline 2-acetoxybenzoate, | 52 |

### Example 35: Protection Against Biotic Stress Caused by Infection of Tomato with Lycopersicon Esculentum Mill Following Fungal Infection of Powdery Mildew, by Spraying Plants

Proceeded in the way as described in Example 34, with the difference that plants were sprayed and not watered. Substances 1-8 in concentrations of 40 mg/l were applied twice by spraying, which resulted in prevention of biotic stress manifested as providing protection against fungal infection.

TABLE 37
Effectiveness in providing protection against powdery mildew
fungus expressed as reduction of leaf area covered by necrotic
spots leaves comparing to this of untreated control plant.
Assessment was made 2 weeks after pathogen inoculation.

| | | Reduction in necrotic |
|---|---|---|
| | | spots (white patches) |
| | Substance | compared to control [%] |
| | Control | 0 |
| 1. | choline 3-chlorosalicylate | 72 |
| 2. | choline 5-chlorosalicylate | 68 |
| 3. | choline 3,5-dichlorosalicylate | 66 |
| 4. | sodium 3-chlorosalicylate | 55 |
| 5. | potassium 3-chlorosalicylate | 54 |
| 6. | calcium 3-chlorosalicylate | 62 |
| 7. | choline 3-chloro-6-fluorosalicylate | 45 |
| 8. | choline 3-chloro-6-bromosalicylate | 49 |
| 9. | choline 3-chloro-6-iodosalicylate | 47 |

1. An aromatic carboxylic acid ionic derivative, wherein anion is defined by a general formula (I) or (II) 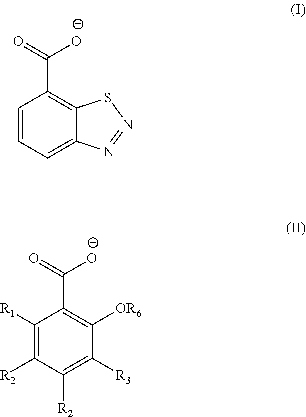
while cation is selected from among M+ and a cation of a general formula (III) 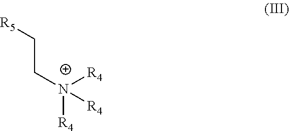
where
R₁ denotes H, F, Br or I;
R₂ and R₃, independently, denote H, F, Cl, Br or I;
R₄ denotes methyl, ethyl, propyl, butyl, decyl or dodecyl;
R₅ denotes hydroxyl, methoxyl, ethoxyl, H, F, Cl, Br, I;
R₆ denotes H, methyl, ethyl or acetyl;
M⁺ denotes lithium, sodium, potassium, calcium or magnesium cation, whereby in case of a divalent cation there are two acid residues per such cation;
for use as a plant stimulant for plant growth regulation and/or regulation of plant metabolic processes.

2. The aromatic carboxylic acid ionic derivative for use according to claim 1, wherein M⁺ denotes sodium or potassium cation, R₁ denotes H, R₂ and R₃, independently, denote H or Cl, R₄ denotes methyl, R₅ denotes hydroxyl, and R₆ denotes H.

3. The aromatic carboxylic acid ionic derivative for use according to claim 1, which is selected from choline 3-chlorosalicylate, choline 5-chlorosalicylate, choline 3,5-dichlorosalicylate and choline benzo[1,2,3]thiadiazole-7-carboxylate.

4. The aromatic carboxylic acid ionic derivative for use according to claim 1, wherein the plant stimulant is selected from a stimulant of root mass growth, a stimulant of fresh and/or dry mass growth of the aboveground parts, a stimulant of increase in shoot number, a stimulant of increase in plant height and/or diameter, a stimulant of increase in yield, a stimulant of increase in the content of health-promoting substances in the green parts, roots and fruit, a stimulant of increase in chlorophyll content, a stimulant of increase in photosynthesis, a stimulant of increase in the efficiency of uptake of active (mineral) substances from soil, a stimulant of increase in fertilizer uptake efficiency.

5. An aromatic carboxylic acid ionic derivative, wherein anion is defined by a general formula (II) 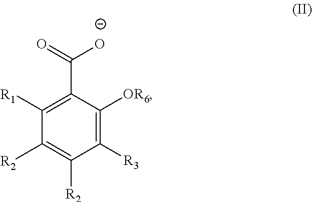
while cation is selected from M+ and a cation of a general formula (III) 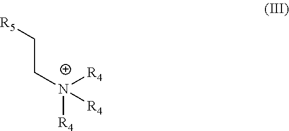
where
R₁ denotes H, F, Br or I;
R₂ and R₃, independently, denote H, F, Cl, Br or I;
R₄ denotes methyl, ethyl, propyl, butyl, decyl or dodecyl;
R₅ denotes hydroxyl, methoxyl, ethoxyl, H, F, Cl, Br, I;
R₆ denotes H, methyl, ethyl or acetyl;
M⁺ denotes a lithium, sodium, potassium, calcium or magnesium cation, whereby in case of a divalent cation there are two acid residues per such cation;
for use as a plant stimulant for preventing the effects of biotic stress caused by activity of viruses, bacteria or fungi.

6. The aromatic carboxylic acid ionic derivative for use according to claim 5, wherein M⁺ denotes sodium or potassium cation, R₁ denotes H, R₂ and R₃, independently, denote H or Cl, R₄ denotes methyl, R₅ denotes hydroxyl, while R₆ denotes H.

7. The aromatic carboxylic acid ionic derivative for use according to claim 5, which is selected from choline 3-chlorosalicylate, choline 5-chlorosalicylate and choline 3,5-dichlorosalicylate.

8. A plant stimulation method involving exposing said plant to a composition containing at least one active ingredient, characterised in that the aromatic carboxylic acid ionic derivative defined in claim 1 is used as the active ingredient.

9. The plant stimulation method according to claim 8, wherein choline 3-chlorosalicylate, choline 5-chlorosalicylate and choline 3,5-dichlorosalicylate or choline benzo[1,2,3]thiadiazole-7-carboxylate is used as the active ingredient of the composition.

10. The plant stimulation method according to claim 8, wherein the composition containing the active ingredient is applied to the plant roots and/or leaves and/or seeds.

11. The plant stimulation method according to claim 8, wherein the composition containing an active ingredient is applied to the plant once every 5 to 21 days.

12. The plant stimulation method of according to claim 8, wherein the composition containing the active ingredient is applied to plants subjected to abiotic stress factors, including drought, too high or too low temperatures or use of herbicides.

13. The plant stimulation method according to claim 8, wherein the composition in form of a solution, preferably an aqueous solution, containing the active ingredient is applied to the plants by spraying or watering.

14. Use of at least one aromatic carboxylic acid ionic derivative defined in claim 1 as at least one active ingredient for production of a composition for plant stimulation by regulation of their height and/or metabolic processes.

15. The use according to claim 14, wherein the aromatic carboxylic acid ionic derivative is selected from choline 3-chlorosalicylate, choline 5-chlorosalicylate, choline 3,5-dichlorosalicylate and choline benzo[1,2,3]thiadiazolo-7-carboxylane.

16. The use according to claim 14, wherein the aromatic carboxylic acid ionic derivative is the sole active ingredient in the composition for plant stimulation.

17. Use of at least one aromatic carboxylic acid ionic derivative defined in claim 5 as at least one active ingredient for production of a composition for plant stimulation by preventing the effects of biotic stress caused by the activity of viruses, bacteria and/or fungi.

18. The use according to claim 17, wherein the aromatic carboxylic acid ionic derivative is selected from choline 3-chlorosalicylate, choline 5-chlorosalicylate and choline 3,5-dichlorosalicylate.

19. The use according to claim 17, wherein the aromatic carboxylic acid ionic derivative is the sole active ingredient in the composition for plant stimulation.
